SEQUENCE LISTING (2 sequences)

SEQ ID NO: 1 - protein, 13 aa, Homo sapiens
TLPPAGVFTRYQK

SEQ ID NO: 2 - protein, 12 aa, Homo sapiens
QEAINELATAMV

(19) United States
(12) Patent Application Publication
(10) Pub. No.: US 2003/0049259 A1
(43) Pub. Date: Mar. 13, 2003

(54) Control of lymphocyte localization by LEEP-CAM activity

(75) Inventors: Michael B. Brenner, Newton, MA (US); Christina M. Parker, Newton, MA (US); Michael P. Schon, Hohendodeleben (DE)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: The Brigham and Women's Hospital, Inc., Boston, MA (US)

(21) Appl. No.: 10/054,714

(22) Filed: Jan. 22, 2002

Related U.S. Application Data
(63) Continuation of application No. 09/552,912, filed on Apr. 20, 2000, now abandoned.
Application No. 09/552,912 is a continuation of application No. PCT/US98/23158, filed on Oct. 30, 1998.
(60) Provisional application No. 60/065,432, filed on Oct. 30, 1997.

(51) Int. Cl. A61K 39/395; A61K 31/00; C07K 16/40

(52) U.S. Cl. 424/146.1; 530/388.26; 514/1

(57) ABSTRACT

A novel cell surface glycoprotein, LEEP-CAM, is disclosed. This invention further provides methods for treating inflammatory disorders in mammals through the administration of compositions which are modulators of LEEP-CAM activity. Antibodies are also disclosed which prevent LEEP-CAM-mediated migration of lymphocytes into epithelial layers of cells.

### RELATED APPLICATIONS

This application is a continuation of U.S. application Ser. No. 09/552,912, filed Apr. 20, 2000, which is a continuation of International Application No. PCT/US98/23158, which designated the United States and was filed on Oct. 30, 1998, which claims the benefit of U.S. Provisional Application No. 60/065,432, filed Oct. 30, 1997. The entire teachings of the above applications are incorporated herein by reference.

### GOVERNMENT SUPPORT

The invention was supported, in whole or in part, by a grant No. NIH A1 38578 from the National Institute of Health. The United States Government has certain rights in the invention.

### BACKGROUND OF THE INVENTION

To carry out immune responses, lymphocytes must be distributed throughout the body and travel between different tissues to come into close proximity with other cell types. In the blood and lymph, lymphocytes circulate as nonadherent cells, while in the tissues, they migrate as adherent cells (Springer, T. A. (1990) Nature 346:425-434). As they move through the body searching for foreign antigens, these cells acquire a tissue specificity based on the environment in which they first encounter their specific antigens and tend to migrate back to that environment.

Adhesion molecules expressed on lymphocytes direct lymphocyte movement to specific microenvironments. This process is called “microenvironment homing” and the first step is the exit of lymphocytes from intravascular spaces into tissues (extravasation). The process of extravasation consists of several steps and involves several molecules in a leukocyte-endothelium interaction.

Following extravasation, the mechanisms of tissue localization and lymphocyte retention after the lymphocytes leave the blood vessels are not well known. Inflammatory skin conditions and other skin disorders are dependent on migration of T lymphocytes into the skin. Interactions between lymphocyte surface receptors and their ligands on epithelial cells critically control migration of leukocytes into sites of inflammation. Understanding the mechanisms through which T cells interact and bind to specific antigens on cells, especially epithelial cells, would be extremely beneficial in understanding skin disorders.

### SUMMARY OF THE INVENTION

The present invention relates to a novel lymphocyte endothelial-epithelial-cell adhesion molecule (hereinafter “LEEP-CAM” or “6F10 antigen”) which is expressed on the surface of epithelial cells and endothelial cells, and is important for T or B cell migration into tissues expressing the 6F10 antigen. LEEP-CAM is expressed on particular epithelia including the suprabasal region of the epidermis, the basal layer of bronchial and breast epithelia, and throughout the tonsillar and vaginal epithelia. It is absent from intestinal and renal epithelia. LEEP-CAM is also expressed on vascular endothelium, especially high endothelial venules (HEV) in lymphoid organs such as tonsil and appendix.

Molecules which inhibit the binding of T lymphocytes to LEEP-CAM, especially antibodies and antibody fragments which bind to the novel LEEP-CAM antigen described herein (or to portions of these sequences) also relate to this invention. In a preferred embodiment, the antibody is a monoclonal antibody (mAb or moAb) which inhibits the adhesion of T lymphocytes to LEEP-CAM and can be used to prevent the migration of T cells into basal skin layers before or during the occurrence inflammatory skin disorders.

Thus, this invention relates to therapeutic compounds which can be used to prevent and/or treat inflammatory conditions. Therapeutic compositions can include small molecule affectors of LEEP-CAM function, particularly inhibitors of LEEP-CAM binding activities with T lymphocytes or LEEP-CAM synthesis. Methods of use or therapy using these compositions are also included in this invention.

This invention also relates to the use of LEEP-CAM antigen and compounds which bind LEEP-CAM for use in diagnostic procedures and in diagnostic kits. The availability of these compounds make it possible to determine the onset of and identify, in particular, various skin disorders mediated by LEEP-CAM. The invention further includes methods of preparing compounds which inhibit LEEP-CAM using the polypeptides and antibodies of the invention.

Thus, this invention provides a system for treating a mammal, especially a human, for diseases and disorders mediated by LEEP-CAM. This approach to preventing and treating skin diseases and autoimmune disorders has several advantages over traditional treatment methods, most importantly, inflammatory reactions can be prevented, decreased or inhibited without depressing the T cell population or other immune system functions.

### BRIEF DESCRIPTION OF THE DRAWINGS

FIGS. 1A-1B are histograms showing that 6F10 mAb blocks the binding between lymphocytes and epithelial cells. Adhesion assays were performed with 16E6.A5 epithelial cells as an adherent monolayer and either ilEL (1A) or PHA blasts (1B) as fluorescent labeled suspension cells. NS.4.1 (isotype matched non-binding antibody), W6/32 (mouse anti-human MHC class I) were used as negative controls, E4.6 mAb, which binds to E-cadherin was used for comparison. Fluorescence units reflecting suspension cell binding to 16E6.A5 adherent cells are shown with error bars representing standard deviations. Experiments were performed with six replicates and repeated three times with similar results. One representative experiment is shown.

FIG. 2 is a histogram showing that the 6F10 mAb inhibits the binding of ilEL to endothelial cell. Human umbilical vein endothelial cells (HUVEC) were grown to confluence as a monolayer in 96 well plates and fluorescence labeled ilEL were used as suspension cells in the adhesion assays. The assays were performed with (a) adhesion buffer without mAb, (b) buffer containing anti-LFA-1 mAb (TS1/22) or (c) buffer containing both anti-LFA-1 mAb (TS1/22) and anti-β1 integrin mAb (4B4). Fluorescence units reflecting suspension cell binding to adherent HUVEC in the presence of control and specific blocking mAb W6/32, E4.6, and 6F10 mAb are shown as means and standard deviations under conditions using the three buffers described in A, B, and C. Experiments were performed with six replicates and repeated three times with similar results. One representative experiment is shown.

FIGS. 3A-3B are histograms showing that the 6F10 mediated adhesion is independent of Ca²⁺ and Mn²⁺. The 3901 ilEL cell line was used as the suspension cells and the breast epithelial cell 16E6.A5 monolayers were used as the adherent cells in a static cell to cell adhesion assay. In FIG. 3A, normal medium (TBS containing 1 mM each of Ca²⁺, Mg²⁺, and Mn²⁺) was used in the incubation and washing steps. In FIG. 3B, medium containing 1 mM Mg²⁺ and 25 mM EGTA was used. NS.4.1 (isotype matched non-binding antibody), W6/32 (mouse anti-human MHC class I antibody) and E4.6 (anti-E-cadherin antibody) were used as control antibodies. Fluorescence units reflecting suspension cell binding to 16E6.A5 adherent cells is shown with error bars representing standard deviations. Each bar represents a mean of six replicates. The experiment was repeated twice with similar results.

FIGS. 4A-4F are histograms showing expression of 6F10 counter-receptor on leukocyte subpopulations. Static adhesion assays between 16E6.A5 cells and (4A) peripheral blood lymphocytes (PBL), (4B) polymorphonuclear cells (PMN), (4C) CD4⁺ PHA blast T cells, (4D) CD8⁺ PHA blast T cells, (4E) freshly isolated tonsillar B cells (4F) activated tonsillar B cells were performed to test the blocking effect of the 6F10 mAb. Each bar represents the mean of six replicates in the adhesion assay and each error bar represents one standard deviation. The result of one experiment is shown. The experiments were repeated at least three times with similar results.

FIGS. 5A-5B are gels showing the 6F10 mAb recognizes an N-glycanase sensitive protein. Immunoprecipitation using the 6F10 mAb was carried out from ¹²⁵I surface labeled cell lines, resolved by SDS-PAGE and visualized by autoradiography. In FIG. 5A, the panel shows the mAb 6F10 immunoprecipitation from cell lysates of epithelial cells (16E6.A5 breast epithelial cell line) and endothelial cells (HUVEC). Lanes 1-3 are immunoprecipitates with NS.4.1 mAb (isotype matched control antibody), W6/32 mAb (mouse anti-human MHC class I) and the 6F10 mAb, respectively, from epithelial cells, while Lanes 4-6 are immunoprecipitates from endothelial cells using the same panel of antibodies. In FIG. 5B, panel shows the 6F10 immunoprecipitate from epithelial cells after N-glycanase digestion. Lanes 1 and 2 are immunoprecipitates with the 6F10 mAb and lanes 3 and 4 used W6/32 mAb. Lanes 2 and 4 are N-glycanase digested immunoprecipitates with the 6F10 and W6/32 mAbs, respectively. The panel in FIG. 5A was resolved in 5-15% gradient SDS-PAGE and the panel in FIG. 5B was resolved on 7.5% SDS-PAGE. Both Panels were resolved under reducing conditions. N-ase: N-glycanase.

FIG. 6 is a graph depicting the effects of 6F10 antibody on ear thickness in mice when the antibody is administered at the time of pro-inflammatory T lymphocyte treatment.

FIG. 7 is a graph depicting the effects of 6F10 antibody on ear thickness in mice when the antibody is administered after treatment with pro-inflammatory T lymphocytes.

FIGS. 8A-8C are photomicrographs depicting the strong expression of LEEP-CAM by endothelia and suprabasal epidermal keratinocytes in normal (8A, left panel) and psoriatic (8B, middle and, 8C, right panel) human skin, and by dermal dendritic cells only in psoriatic skin (middle and right panel). Five μm cryostat-cut sections were stained by the ABC-peroxidase method with the 6F10 mAb. In the right panel, the dermo-epidermal junction is indicated by a dashed line. Scale bars=20 μm.

FIGS. 9A-9B are photomicrographs (9A) and a histogram (9B) depicting the adhesion of PHA-blasts to suprabasal epidermal layers of psoriatic skin, but not to the basal epidermal layer or to the dermal compartment, is mediated by LEEP-CAM. FIG. 9A represents 5 μm cryostat-cut sections of human psoriatic skin which were incubated with medium only (left panel), the isotype-matched N-S.4.1 mAb (middle panel), or the 6F10 mAb (right panel). PHA-blasts were allowed to adhere to the sections for 35 minutes as outlined in the Exemplification. Sections then were fixed and stained with hematoxylin. Scale bar=20 μm. FIG. 9B represents PHA-blasts bound to the basal or suprabasal layers of normal or psoriatic skin as indicated were quantitated per mm skin. Average counts and standard deviations from three independent experiments are depicted.

FIGS. 10A-10C are photomicrographs (10A, 10B) and a histogram (10C) showing that LEEP-CAM mediates T cell migration into monolayers of immortalized human keratinocytes. In FIG. 10A, Modified Boyden-chambers were equipped with polycarbonate filters coated with a monolayer of HaCaT cells on the undersurface. PKH26-labeled activated T cells (PHA-blasts) were seeded into the upper compartment and allowed to migrate for 3.5 hours. Filters then were washed and frozen in O.C.T. 5 μm cryostat-cut sections were stained with the 6F10 mAb by the indirect immunofluorescence technique using a FITC-conjugated second antibody. LEEP-CAM expression then was visualized by fluorescence microscopy using a green filter (upper panel) and immigrated T cells were visualized within the same field using a red filter (lower panel). In FIG. 10B, after migration of PKH26-labeled T cells, filters were washed, fixed, and mounted onto slides. Migrated T cells were visualized in a fluorescence microscope using a red filter. Left panel: uncoated and untreated filter, second panel: filter coated with a HaCaT-monolayer and pre-incubated with culture medium; third panel: HaCaT-coated filter pre-incubated with the N-S.4.1 mAb; right panel: HaCaT-coated filter incubated with the 6F10 mAb. FIG. 10C shows PKH26-labeled T cells which migrated into the HaCaT-coated polycarbonate filters and were quantitated per mm². Migrated cells in uncoated filters (left column), HaCaT-coated filters incubated with culture medium (second column), HaCaT-coated filters treated with the N-S.4.1 mAb (third column), and HaCaT-coated filters treated with the 6F10 mAb are shown. Values shown represent counts and standard deviations from three independent chambers. * indicates p-0.000 I and ** indicates p=0.003.

FIGS. 11A-11B are photomicrographs showing that LEEP-CAM is strongly expressed in organotypic cultures of human keratinocytes and mediates binding of activated T cells to the viable epidermal layers in these cultures. In FIG. 11A, organotypic cultures of normal human keratinocytes were generated on top of a collagen/fibroblast dermis equivalent as outlined in the Exemplification. To show orthokeratinization and orthotopic expression of differentiation markers, 5 μm cryostat-cut sections of these cultures were stained with mAbs against keratin K1/10 (left panel), involucrin (second panel), keratin K5 (third panel), gp80 (fourth panel), or LEEP-CAM (right panel). Dashed lines indicate the location of the viable cell layers between the dermis equivalents and the cornified layer. In FIG. 11B, PKH26-labeled PHA-blasts were allowed to adhere to 5 μm cryostat-cut sections of organotypic cultures of normal human keratinocytes pre-incubated with the isotype-matched control mAb N-S.4.1 (left panel) or the 6F10mAb (right panel). Sections then were washed, fixed, and stained with hematoxylin. Sequential sections of the same area are shown. The dermo-epidermal junction is indicated by the dashed line. Bound T cells are represented by dark blue dots. T cell binding to the dermal compartment is not affected by the 6F10 mAb. Scale bars=20 μm.

FIGS. 12A-12D are photomicrographs (12A-12C) and a histogram (12D) showing that activated T cells migrate into organotypic cultures of normal human keratinocytes and the 6F10 mAb dramatically inhibits this experimental epidermotropism. In FIG. 12A, the undersurface of an organotypic culture of normal human keratinocytes was stained with the 6F10 mAb by the indirect immunofluorescence method after the dermis equivalent was removed. Please note the intact cobble-stone pattern of the basal keratinocytes and the strong binding of the 6F10 mAb. In FIG. 12B, after migration of PKH26-labeled PHA-blasts into organotypic cultures of human keratinocytes, the cultures were washed and snap frozen in liquid nitrogen as outlined in the exemplification. To confirm T cell migration into the epidermoids, 5 μm cryostat-cut sections were examined in a fluorescence microscope using a red filter. Migrated T cells are visualized as bright red dots. The location of the basement membrane and the border between viable and cornified epidermal layers are indicated by a dashed line. Scale bar=20 μm. T cells migrated only in the viable epidermal layers. In FIG. 12C, whole mounts of organotypic cultures of normal human keratinocytes are shown after migration of PKH26-labeled PHA-blasts. Organotypic cultures were incubated prior to the T cell migration with culture medium, the N-S.4.1 control mAb, or the 6F10 mAb as indicated. Labeled cells were visualized in a fluorescence microscope using a red filter. The low-power photomicrographs demonstrate the reduced number of migrated T cells and the high-power photomicrographs show the lack of T cell processes in 6F10 treated cultures. Scale bars=20 μm. In FIG. 12D, T cells which migrated into organotypic cultures of normal human keratinocytes were quantitated per mm² Values shown represent average counts and standard deviations from three independent experiments. Organotypic cultures were incubated with culture medium or mAbs prior to T cell migration as indicated.

FIG. 13 is a gel showing the nine (1/9 to 9/9) anti-LEEP-CAM monoclonal antibodies recognize glycoproteins having a relative mobility of 70 kDa and 100 kDa from 16E6.A5 epithelial cells.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

This invention relates to a novel endothelial and epithelial cell adhesion molecule, LEEP-CAM, which is expressed in a variety of normal epithelial and endothelial tissues of mammals, especially humans. This invention further relates to compositions (LEEP-CAM antagonists) which inhibit the adhesion of T or B lymphocytes to LEEP-CAM including, but not limited to, antibodies and antibody fragments. The term “LEEP-CAM antagonist” refers to a compound which interferes with or inhibits the interaction between LEEP-CAM and T cells, in particular, LEEP-CAM mediated adhesion of T cells. In particular, monoclonal antibodies against LEEP-CAM (e.g. 6F10 mAb) can block the adhesion between epithelial/endothelial cells and activated lymphocytes.

Lymphocyte adhesion within the epithelium is important in host defense. Except for those infectious agents that gain direct access to the body via trauma or arthropod vectors, most infectious microorganisms must interact with the mucosal or cutaneous epithelium in order to invade the host. Therefore, immune reactions in the epithelium are one of the first lines of defense against infections from the environment. The epithelium is also the origin of most adult cancers such as of the breast, lung, colon and uterine cervix. Lymphocytes in the epithelium may play important roles in both defending against infection and in tumor surveillance. Intraepithelial lymphocytes (EL) represent a special subpopulation of lymphocytes, composed mainly of T cells, that are resident in epithelial compartments. They occupy a unique anatomical site in direct contact with epithelial cells, enabling them to respond to infectious and malignant challenges within the epithelium. Due to the large surface area of epithelial organs, there are as many lymphocytes in the epithelium as in the organized peripheral lymphoid organs. Yet, little is known about the adhesive interactions between lymphocytes and epithelial cells. A few specific adhesion molecules mediating EEL adhesion to epithelium have been delineated. Intestinal EEL express the α^{E}β7 integrin which mediates specific adhesion to E-cadherin expressed on epithelial cells. These molecules could mediate cell to cell interactions between T cells and epithelial cells that stabilize the retention of lymphocytes in the epithelium. LEEP-CAM is a newly identified molecule that is expressed on selected epithelial cells and on endothelial cells, and is involved in the binding of lymphocytes to these tissues.

The LEEP-CAM antigen mediates the homing of lymphocytes to skin epithelium (epidermis) and the endothelium. Blocking the epithelia and/or endothelia with 6F10 mAb through local administration or systemic administration can block adhesion between lymphocytes and epithelial cells, thus preventing or decreasing skin inflammation. Thus this invention further relates to methods of preventing the adhesion of T or B lymphocytes to LEEP-CAM, especially methods which do not deplete the concentration of T lymphocytes in the body of a mammal.

The distribution of LEEP-CAM is different from all other known adhesion molecules. It is a lymphocyte endothelial-epithelial-cell adhesion molecule which is expressed on suprabasal epithelial cells in the skin, some epithelial cells at other sites, freshly isolated monocytes, dendritic-appearing cells which co-express MHC class II in psoriatic skin, and on some endothelial cells such as high endothelial cells in the tonsil and endothelial cell in psoriatic and uninflammed skin.

Useful inhibitors of T cell adhesion to the 6F10 antigen would block specific adhesion sites on LEEP-CAM or block a specific ligand on a T cell which binds to LEEP-CAM. These antagonists would thus prevent inflammatory reactions resulting from migration of T cells into suprabasal epithelial tissues.

There are a multitude of different diseases which involve T cells as critical components. These include autoimmune diseases and infections, but also T cell-derived tumors (e.g. cutaneous lymphomas). In these diseases, T cells exert most of their pathogenic effects within the parenchyma of tissues (cytokine secretion, cytotoxicity, migration, etc.). While T cell extravasation and its importance for the localization of T cells is a well-studied field, very little is known about the migration of T cells within the parenchymatous organs. In particular, very little is known about adhesive interactions of T cells with tissue cells which mediate tissue selectivity of T cell localization.

Skin diseases present an example which involves T cell migration. Once T cells have extravasated, they migrate into both the connective tissue and the epidermis. This is in common in many skin disorders, ranging from inflammatory reactions in autoimmune diseases (e.g. psoriasis and lichen ruber) to malignant tumors (e.g. cutaneous T cell lymphomas). In these conditions, T cells migrate a relatively much longer distance within the connective tissue and the epidermis than they cover transmigrating the endothelial wall. Especially epidermotropism is very poorly understood, because ligands for many well-known T cell adhesion molecules are not expressed in this site. These include ligands for T cell integrins (collagen, laminin, fibronectin, ICAM-1, VCAM) and selecting. Thus, it is currently unclear how T cells localize to suprabasal layers of the epidermis. The identification of LEEP-CAM, its expression pattern and its in vitro properties indicate it is a receptor for T cell epidermotropism.

Studies of naive and memory T lymphocytes show differences in these two subsets of T cells regarding trafficking and recirculation. Naive lymphocytes are continually produced in the bone marrow and the thymus and exit the circulatory system into the lymph nodes where they can encounter foreign antigens, undergo activation, and differentiate phenotypically into effector and memory T cells. Naive cells which do not encounter foreign antigens and therefore do not change phenotypically, simply pass through the lymph nodes without being activated and “recirculate” between tissue and blood.

The memory T cells eventually drain via efferent lymphatic ducts back to the bloodstream but do not preferentially return to the lymph nodes. The activated lymphocytes (memory cells) generally express higher levels of tissue specific adhesion molecules and are capable of homing to extralymphoid sites of inflammation, including epithelial tissues. Memory cells traffic to their effector sites to perform specific immune functions. Among the important target sites for memory cells are the epithelial organs, including the wet mucosal surfaces (alimentary, genito-urinary and respiratory tracts) and the skin.

The mechanisms by which T cells can be transported to epithelial sites, including gut and skin, has been the subject of intense investigation. For tissue-specific lymphocytes to reach their target microenvironments, lymphocytes first have to extravasate from the blood vessels in the target organ, then migrate and adhere to the destination microenvironment. Adhesion molecules on endothelium cells facilitate the recruitment of lymphocytes expressing particular counter-receptors into tissue stroma. After entering the tissue, lymphocytes must be guided and localized by adhesion molecules expressed on tissue stroma cells, including epithelial cells. Compared with leucocyte-binding molecules on the endothelium, little is known regarding epithelial molecules mediating leukocyte binding.

Identification of a Monoclonal Antibody Inhibiting Lymphocyte Adhesion to Epithelial and Endothelial Cells

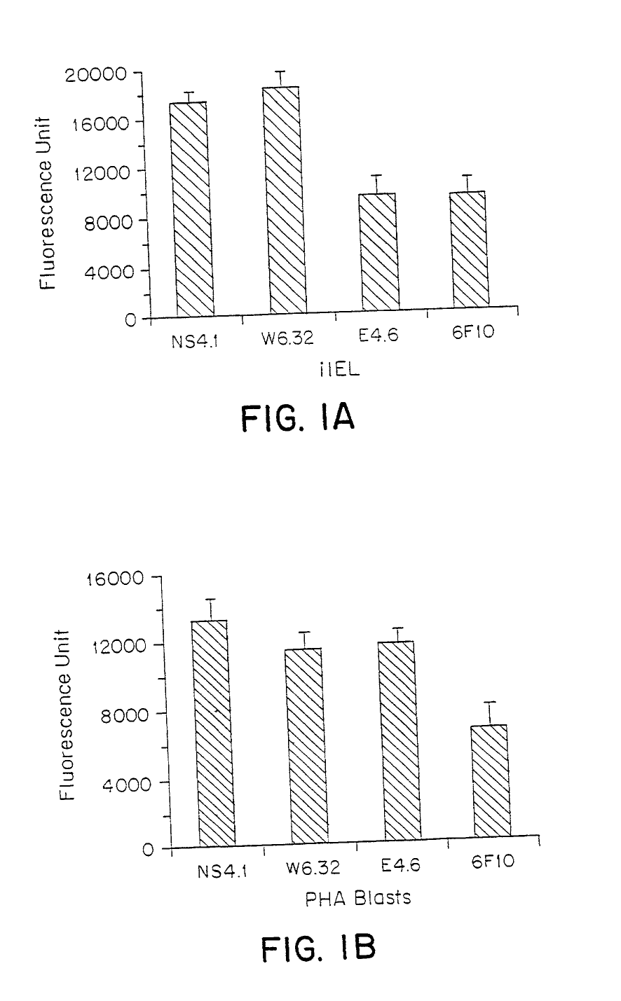

To identify adhesion molecules involved in lymphocyte binding to epithelial and endothelial cells, BALB/cJ mice were immunized with the 16E6.A5 cell line derived from human breast epithelium and produced monoclonal antibodies. The hybridoma supernatants were screened to identify those which blocked the binding of in vitro cultured T cells to 16E6.A5 epithelial cell monolayers in static cell-to-cell adhesion assays. One mAb, designated 6F10, stained the immunizing epithelial cell line and blocked the adhesion between T cells and epithelial cells effectively and was selected for further study. The 6F10 mAb ascites reproducibly blocked the binding of T cells to epithelial cell monolayers by approximately 60%, similar to the degree of blocking observed with anti-E-cadherin mAb, E4.6 (FIG. 1A). T cell adhesion to epithelial cells can be mediated by the T cell integrin, α^{E}β7, and epithelial cell E cadherin (Cepek, K. L., et al. (1994) Nature 372:190). To determine if the 6F10 antigen was involved in α^{E}β7/E-cadherin adhesion, studies were performed using PHA blast T cells that lack significant levels of α^{E}β7 expression. As expected, when short term PHA stimulated T cell lines were examined, adhesion to epithelial cell monolayers was not blocked by mAb E4.6 against E-cadherin. Nevertheless, the 6F10 mAb still significantly blocked PHA blast T cell adhesion to epithelial cells by 50% in comparison to negative control mAb N-S.4.1 or W6/32 (FIG. 1B). Thus, 6F10 antigen dependent adhesion between T cells and epithelial cells is distinct from that mediated through the α^{E}β7 integrin-E cadherin interaction.

Tissue Distribution of the 6F10 Antibody Staininq

Flow cytometric analysis (FACS) and immunoperoxidase tissue staining were used to determine the cellular distribution of the 6F10 antigen expression. First, a panel of cultured human cell lines was analyzed by flow cytometry. As shown in Table 1, several epithelial derived cell lines including 16E6.A5 (breast origin), A431 (epidermal squamous cell carcinoma), and primary cultures of keratinocytes were stained brightly with mean fluorescence intensities (MFI) of 448, 445, and 751, respectively. Other epithelial cell lines were stained weakly (T84) or were negative (293T). Cells of endothelial origin, including HUVEC (endothelial cell primary culture), ECV304, a spontaneously transformed HUVEC cell line, and HMEC-1, a transformed microvascular endothelial cell line all stained with the 6F10 mAb with MFIs of 641, 69, and 165, respectively. Thus several cell lines of endothelial or epithelial origin expressed the 6F10 antigen. In addition, platelets (MFI 161) and freshly isolated blood monocytes (MFI 308) were stained with the 6F10 mAb. These freshly isolated blood monocytes lost expression of the 6F10 antigen during 3 days of in vitro culture. All other cell lines of myelomonocytic or lymphocytic lineages lacked reactivity with the 6F10 mAb (Table 1). FACS analysis of cell lines stained with 6F10 antibody

| Type | Cell line | Staining | MFI* |
|---|---|---|---|
| Epithelial | 16E6.A5 (breast) | Positive | 448 |
| | A431 (epidermis) | Positive | 445 |
| | primary keratinocyte | Positive | 751 |
| | T84 (colon) | Weakly positive | 32 |
| | 293T (embryonic kidney) | Negative | 3 |
| Endothelial | HUVEC | Positive | 641 |
| | CDC.HMEC-1 | Positive | 165 |
| | ECV304 | Positive | 69 |
| Myelo-monocytic | Fresh PB monocytes | Positive | 308 |
| | Cultured monocytes** | Negative | 9 |
| | ThP1 | Negative | 4 |
| | U937 | Negative | 3 |
| | HL60 | Negative | 5 |
| Platelets | PB platelets | Positive | 161 |
| Lymphocytic | JY (B cell lymphoblastic) | Negative | 4 |
| | PB lymphocytes | Negative | 7 |
| | ilEL (3901) | Negative | 7 |

*MFI: mean fluorescence intensity
**Three day culture in RPMI media

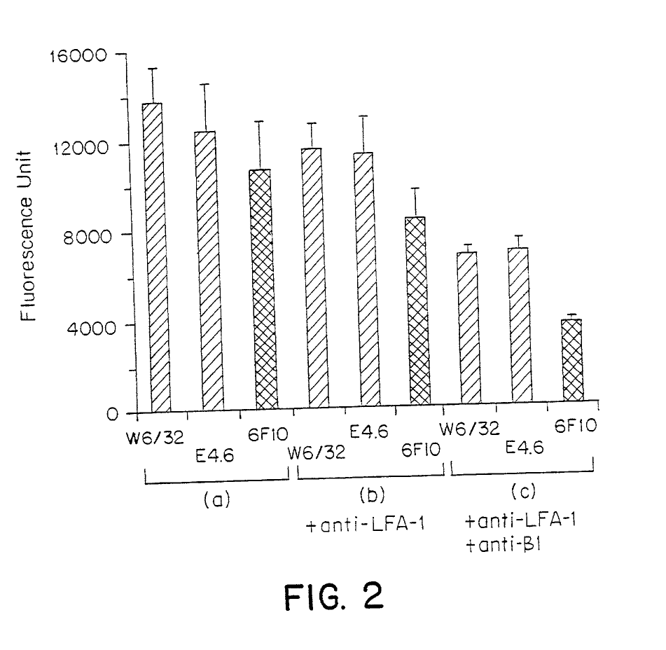

To evaluate the 6F10 antigen expression in vivo, immunoperoxidase staining of human tissue sections was performed. In this analysis, the 6F10 mAb stained the basal layer (B) of breast ductal epithelium, the suprabasal layer (Sb) of stratified epithelium in skin, the basal and suprabasal layers of tonsillar epithelium, the basal cells (B) of bronchiolar epithelium and the vaginal and endometrial epithelia of the uterine cervix. However, epithelial tissue expression of the 6F10 antigen was selective since the columnar epithelium (Ep) of intestine and the cuboidal epithelium of renal tubules were negative. Endothelial expression also was noted with prominent staining of high endothelial venule (HEV) endothelium in lymphoid tissues such as appendix, tonsil mesenteric lymph node and peripheral lymph node. This staining was intense on the lumenal side of the HEV where endothelial cell-lymphocyte interactions occur. Scattered cells with a dendritic appearance typical of tissue macrophages (M) were stained in the lamina propria (Lp) just under the epithelium of the appendix and the large intestine. The 6F10 Mab Inhibits the Adhesion of Lymphocytes to Endothelial Cells The 6F10 mAb was identified based on its ability to block T cell adhesion to epithelial cells. Since the 6F10 antigen also was expressed on endothelia (Table I), adhesion assays between ilEL and monolayers of human umbilical vein endothelial cells (HUVEC) were performed. The binding of lymphocytes and HUVEC is known to be mediated by several adhesion molecule-counter-receptor interactions including LFA-1 (α^{L}β₂)-ICAM1, 2, and VLA-4 (α⁴β₁)-VCAM-1. With these other adhesive interactions intact, the 6F10 mAb inhibited T cell-HUVEC adhesion by only 20% compared to the level of adhesion seen using control mAb against MHC class I (FIG. 2,a). The inhibition became more evident when the lymphocytes were pre-incubated with anti-LFA-1 mAb, TS1/22 (FIG. 2,b) and was readily observed when the lymphocytes had been preincubated in the presence of both anti-LFA-1 mAb, TS1/22 and anti-β1 integrin mAb, 4B4, with more than 50% inhibition of binding by the 6F10 mAb compared to control mAb (FIG. 2,c). As expected. the mAb E4.6 against E-cadherin had no significant effects in these experiments, even in the presence of other anti-integrin antibodies, as E-cadherin is not expressed by HUVEC. Thus, 6F10 antigen binding contributes to lymphocyte adhesion to endothelial as well as to epithelial cell substrates.

Divalent Cation Requirement for 6F10 Mediated Adhesion

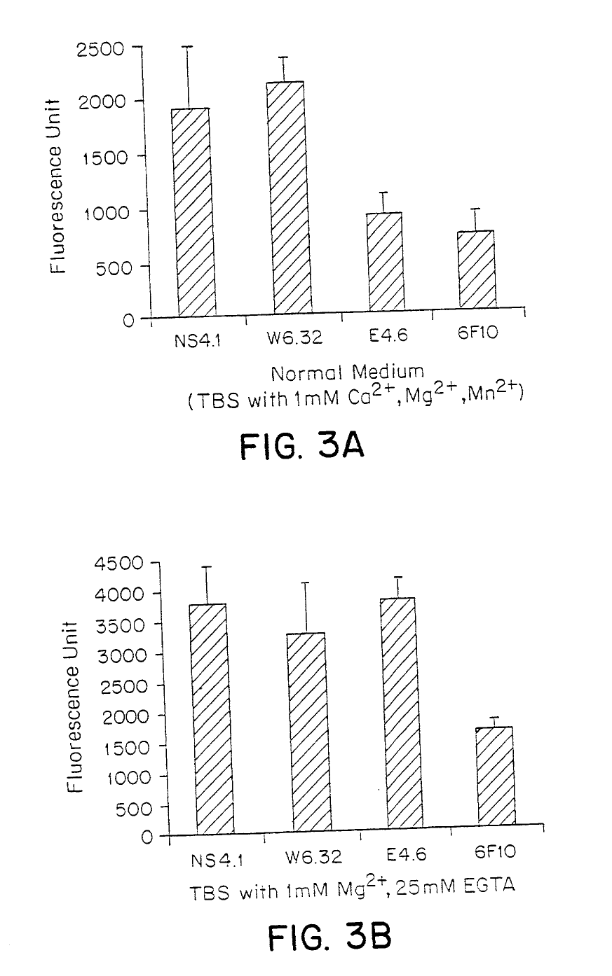

The divalent cation requirements for the 6F10 antigen mediated lymphocyte-epithelial cell adhesion were characterized and it was determined that the 6F10 antigen mediated binding is not dependent on Ca²⁺ or Mn²⁺. The 6F10 mAb blocked the binding of ilEL T cells to epithelial cell monolayers by approximately 60% when compared with control mAb in the presence of 1 mM Ca²⁺, Mg²⁺ and Mn²⁺ (FIG. 3A). Monoclonal antibody E4.6 against E-cadherin also blocked the binding of Eα^{E}β₇⁺ ilEL T cells to 16E6.A5 cell monolayers to levels that were similar to that noted for the newly developed 6F10 mAb (FIG. 3A).

Static adhesion assays between 16E6.A5 epithelial cells and ilEL were also performed in medium without Ca²⁺ and Mn²⁺. To ensure the integrity of epithelial cell monolayers, 1 mM of Mg²⁺ was added to the adhesion medium along with 25 mM of EGTA, which has a 10⁵ fold greater affinity for Ca²⁺ than for Mg²⁺ and Mn²⁺ in the adhesion medium, the blocking Mg²⁺. In the absence of Ca²⁺ and Mn²⁺ in the adhesion medium, the blocking effects of the anti-E-cadherin mAb E4.6 decreased from 55% to 0% (FIGS. 3A, 3B E4.6, compared with W6/32), as expected based on the requirements for activation of integrin α^{E}β7 by Mn²⁺ and E-cadherin for calcium in adhesion. In contrast, blocking by the 6F10 mAb was not significantly affected by the removal of Ca²⁺ and Mn²⁺. Blocking was 60% and 50% in the presence and absence of Ca²⁺ and Mn²⁺ (FIG. 3A, 3B, 6F10, compare with W6/32). Thus, the adhesion mediated by the 6F10 antigen, in contrast to the adhesion mediated by (α^{E}β7-E-cadherin, was not dependent on the presence of Ca²+and Mn²+. Assays to determine the role of Mg²⁺ in adhesion were not conclusive since the monolayer of epithelial cells that served as the adhesion substrate was not adequately maintained in the absence of Mg²⁺.

Leukocyte Subpopulations that Express the 6F10 Counter-receptors

The counter-receptor for the 6F10 antigen has not yet been determined. To identify the cells that can bind to epithelial cells through 6F10 antigen recognition, several cell types were tested as suspension cells in adhesion assays using 16E6.A5 epithelial cell monolayers as adherent cells. The cells tested included ilEL, peripheral blood lymphocytes, PHA-stimulated T cell blasts (PHA blasts) and their CD4⁺ or CD8⁺ subsets, freshly isolated and activated B cells and polymorphonuclear cells (PMN).

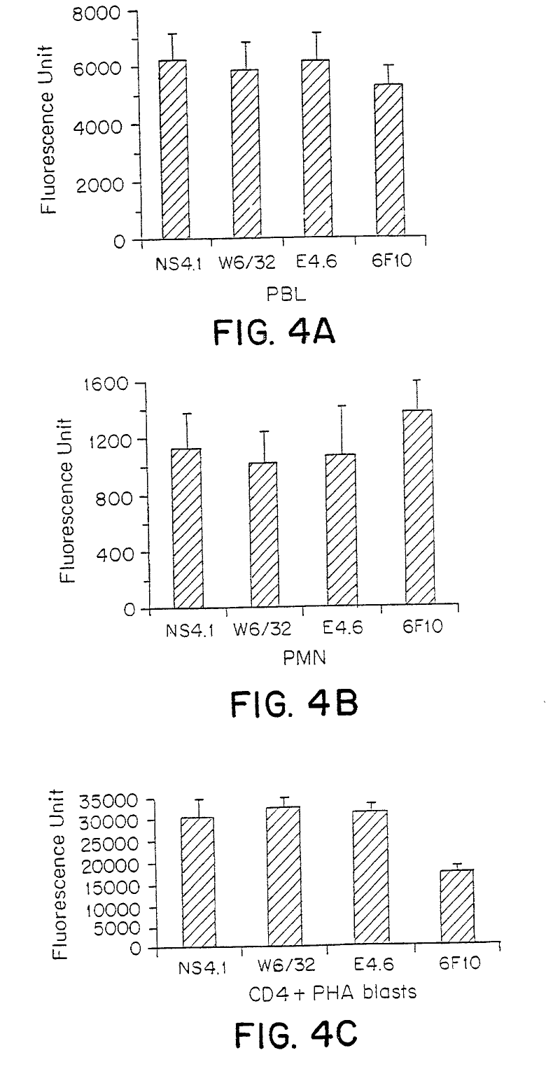

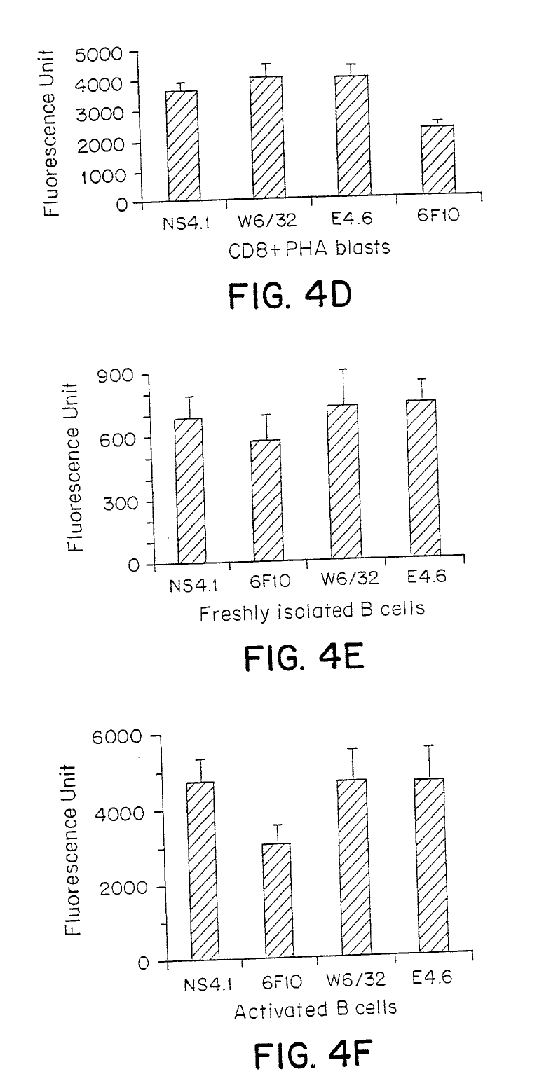

ilEL and PHA blast T cells bind 16E6.A5 cells in a 6F10-dependent manner (FIGS. 1A, 1B). To determine if freshly isolated peripheral blood lymphocytes (PBL) also were capable of binding 16E6.A5 epithelial cells in a 6F10 dependent manner, monocyte depleted peripheral blood mononuclear cells (PBMC) were used as the suspension cells in the adhesion assays. In comparison with the paired experiment with ilEL in which 60% of the binding could be blocked by the 6F10 mAb, fresh PBL binding could be blocked by only about 10% with the 6F10 mAb (FIG. 4A, 6F10 and W6/32, p>0.05). CD4⁺ and CD8⁺ subpopulation of freshly isolated PBL were also tested for 6F10antigen mediated binding in adhesion assays. Both CD4⁺ and CD8⁺ PBL showed minimal 6F10 mAb blockable adhesion indicating that neither the whole population of fresh PBLs nor the CD4⁺/CD8⁺ subpopulations of PBL had significant 6F10 mAb blockable binding to epithelial cells. Similarly, freshly isolated PMN also showed no blockable adhesion to the epithelial cells (FIG. 4B) when compared with the 60% blocking in a paired experiment with ilEL as the suspension cells.

Since PHA activated PBL adhesion to epithelial cells was 6F10 antigen dependent (FIG. 1B), potential differences in the CD4⁺ or CD8⁺ subpopulations of PHA blast T cells in adhesion to epithelial cells were examined. Both the CD4⁺ and CD8⁺ PHA stimulated lymphoblasts bound epithelial cells similar to the mixed population of PHA blasts and could be blocked with the 6F10 mAb by about 50% when compared to blocking with control mAb (FIGS. 4C, 4D). Thus the 6F10 antigen mediated binding contributes comparably to CD4⁺ and CD8⁺ populations of PHA blasts in binding to epithelial cells.

B cells also were tested for their ability to bind 16E6.A5 epithelial cells. Although slight decreases in the binding of freshly isolated B cells to 16E6.A5 epithelial cells were seen in the presence of the blocking 6F10 mAb, these differences were not significant when compared to mAb NS.4.1, the isotype matched control or mAb w6/32, the cell binding control (FIG. 4E). However, B cells activated with the B-cell specific mitogen, formalin-treated SAC, bound 16E6.A5 cells in a 6F10 dependent manner (FIG. 4F) such that the binding could be blocked with the 6F10 mAb by 40% when compared to blocking with control mAbs. Similarly, binding of B-lymphoblastoid cell lines to 16E6.A5 cells was also blocked by the 6F10 mAb.

Thus, PHA lymphoblasts, activated B cells, as well as ilEL cell lines bind epithelial cells in a 6F10 dependent fashion that is independent of adhesion mediated through the α^{E}β7 integrin-E-cadherin interaction. The suspension cells (ilEL, PBL, PHA blasts, B cells, PMN) tested in these adhesion assays did not express the 6F10 antigen themselves as seen by flow cytometric analysis (Table 1) and therefore presumably express a heterophilic counter-receptor for the 6F10 antigen.

The 6F10 Mab Immunoprecipitates an N-glycanase Sensitive Molecule Distinct from Other Known Cell Adhesion Molecules

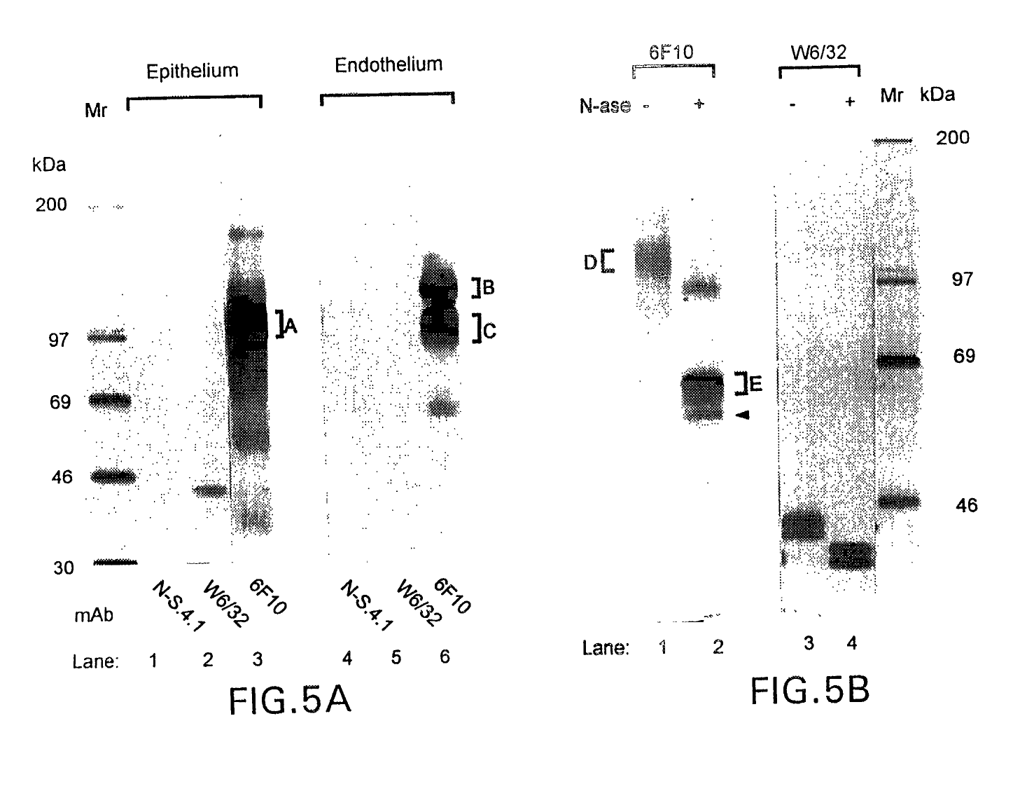

After cell surface labeling with ¹²⁵I, 16E6.A5 epithelial cells or HUVEC were solubilized in TBS containing 1% TX100 and 0.5% DOC, immunoprecipitated with the 6F10 mAb and resolved in SDS-PAGE (FIG. 5A). The immunoprecipitated radiolabeled species resolved as a major broad band having a mean relative mobility of 105 kDa from epithelial cells (FIG. 5A, lane 3, bracket A) and 100 kDa and 145 kDa from endothelial cells (FIG. 5A, lane 6, brackets B and C). After treatment with N-glycanase, the radiolabeled species from epithelial cells (105 kDa, FIG. 5B, lane 1, bracket D) decreased in apparent molecular weight to approximately 65 kDa (FIG. 5B, lane 2, bracket E) with several more weakly labeled species, the smallest of which was 55 kDa (FIG. 5B, lane 2, arrow head). The apparent molecular weights of immunoprecipitates were not changed after O-glycanase digestion.

Based upon these biochemical studies, the 6F10 antigen appears to be a glycoprotein containing approximately 40 kDa of asparagine (N)-linked additions. These biochemical features and the prominent expression on selected epithelia and endothelia distinguishes the 6F10 antigen from other known cell adhesion molecules to which lymphocytes bind.

Protein Isolation

6F10 antigen was purified in a two step procedure using an immunoaffinity column followed by 2-dimensional IEF/SDS-PAGE separation. The putative protein was transferred to PVDF membrane, digested with trypsin and submitted for amino acid determination. The derived peptides were separated with HPLC and sequenced using an Applied Biosystems model 470 A gas phase sequencer equipped with a model 120A phenylhydantoin amino acid analyzer. Two unique internal amino acid sequences, Peptide No. 1 and Peptide No. 2, were obtained that have no matching sequence in the protein databases:

| | |
|---|---|
| Peptide No. 1 | |
| T(L)PPAGVFYQ(K) | SEQ ID NO:1 |
| Peptide No. 2 | |
| Q-(E)(A)INEL(A)(T)(A)(M)(V). | SEQ ID NO:2 |

The amino acids were designated by the single letter codes. Letters with parentheses represent low signals. Other letters represent signals with high confidence.

Involvement of LEEP-CAM in the Pathogenesis of Skin Disorders such as Psoriasis.

Psoriasis, one of the most common skin diseases which affects approximately 2% of the population, is thought to be a T-cell mediated autoimmune disease (Barker, J. N. W. N. (1994) Bailliere 's Clin. Rheumatol. 8:429-437; Christophers, E. (1996) Int. Arch. Allergy Immunol., 110: 199-206). Although there is evidence which suggests a primary pathogenic role of T-lymphocytes in psoriasis (Gottlieb, J. L., et al. (1995) Nature Med., 1:442-447) there has been no direct demonstration of this in human patients. Dysregulated T-cells in mice are able to induce psoriasis-like tissue alterations (Schön, M. P., et al. (1997) Nature Med., 3:183-188). Evidence is accumulating suggesting a compartmentalization of infiltrating T-lymphocytes themselves within psoriatic skin. For example, CD8⁺ T-cells localize primarily to the epidermis, whereas CD4⁺ T-cells are predominant within the dermis, and there appears to be a preferential localization of specific V-gene bearing T-cells within the epidermis. Thus, it appears that different pathways of adhesive interactions exist governing T-cell localization within the cutaneous microenvironment. It is likely that different sets of adhesion molecules expressed on either T-lymphocytes or components of the skin are critically involved in this selective process. However, the molecular interactions leading to selective localization and activation of T-cells in the different skin compartments are still poorly understood. The study of LEEP-CAM antigen which mediates interactions between T-cells and other cell types involved in the psoriatic disease process, however, has shed light on key steps of the pathogenesis of this common disease.

The antibody recognizing LEEP-CAM antigen, 6F10, has been identified by its ability to inhibit adhesive interactions between T-cells and both epithelial and endothelial cells in vitro. LEEP-CAM is expressed on both endothelial and epithelial cells, and use of 6F10 demonstrated its involvement in the several steps of the pathogenesis of skin disorders such as psoriasis.

Activated T-cells express a variety of receptors that can potentially mediate transmigration through the endothelium (e.g., LFA-1 binds to endothelial expressed ICAM-1), the dermis (e.g., various VLA-receptors bind to collagen) and the basal layer of the epidermis (e.g. to laminin and collagen IV). In contrast, none of the known receptors is expressed in suprabasal layers of the epidermis, yet T-lymphocytes are found in this compartment in psoriatic lesions.

It was reasoned that LEEP-CAM could guide T-cells to the intraepidermal compartment and therefore play an important role in some aspects of the pathogenesis of skin disorders such as psoriasis. The distribution of LEEP-CAM in normal and psoriatic skin was determined and LEEP-CAM was tested for its ability to mediate adhesive interactions within both skin conditions. In addition, the expression of LEEP-CAM was assessed in a recently described T-cell mediated mouse model of psoriasis. To confirm the in vitro binding studies, the murine model was utilized to perform in vivo analyses of cutaneous T-cell localization during disease development.

The results demonstrated that LEEP-CAM is expressed on endothelial cells and suprabasal keratinocytes in normal and psoriatic skin and on “lining macrophages” exclusively in psoriatic skin.

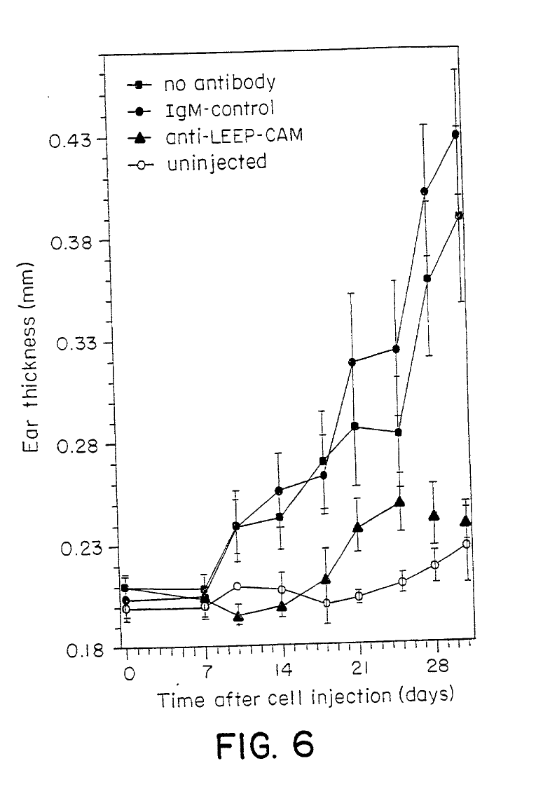

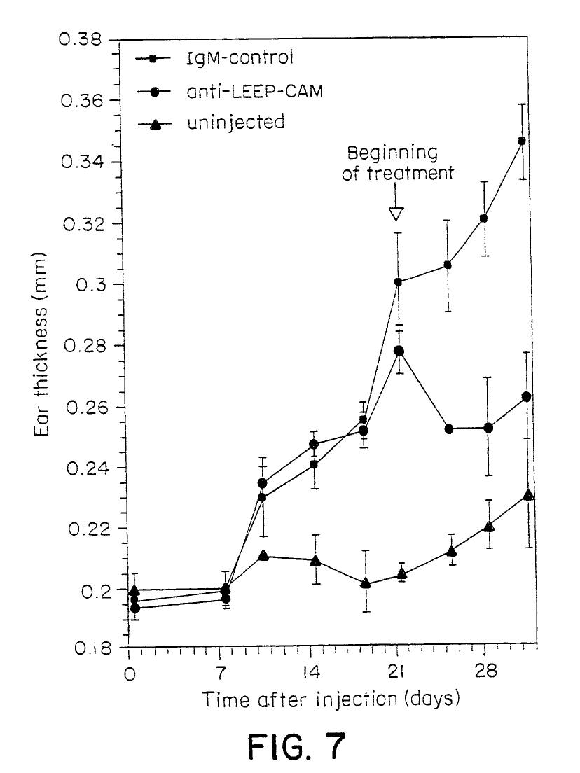

Prior experiments had demonstrated that the transfer of splenocytes isolated from integrin aE deficient mice into severe combined immunodeficient mice resulted in inflammatory skin lesions. To determine if the 6F10 antibody would alleviate these inflammatory skin lesions, a treatment study was performed. Scid mice were injected with between 2.0 and 2.5×10⁷ splenocytes isolated from aE deficient mice. Animals were treated either with F(ab) fragments generated from the 6F10 antibody or with fragments from a non-cell binding IgM control monoclonal antibody, using 0.2 mg of fragments administered intraperitoneally every 2 days for the duration of the study. Treatment was initiated either 16 hours prior to the injection of the pro-inflammatory population of T lymphocytes, or at 14 days or 21 days after cell transfer. The severity of skin inflammation was evaluated by measuring the ear thickness with an ear thickness gage, in the treated and untreated groups of animals. As shown in FIGS. 6 and 7, the 6F10 antibody substantially reduced the increased ear thickness observed in these animals, when administered either at the time of cell transfer (FIG. 6) or when lesions already had developed (FIG. 7). This observation suggests that 6F10 mAb or other inhibitors of LEEP-CAM mediated T cell adhesion are useful as a treatment for inflammatory skin lesions.

Expression of LEEP-CAM in Normal and Psoriatic Human Skin

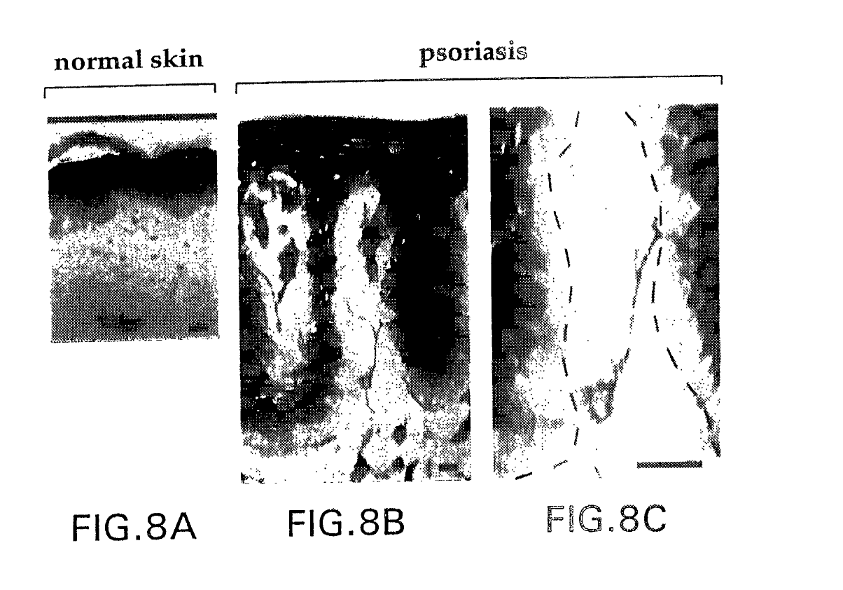

To assess LEEP-CAM expression in human inflamed conditions, LEEP-CAM was analyzed by immunohistochemistry in normal and psoriatic human skin. As shown in FIG. 8A, left panel, suprabasal keratinocytes and dermal endothelial cells in normal human skin (4/4) strongly express LEEP-CAM. Similarly, suprabasal keratinocytes of the hyperproliferative psoriatic epidermis, and endothelial cells of the numerous and dilated dermal blood vessels in psoriatic lesions show strong reactivity with the 6F10 MAb (FIG. 8B, middle panel). Interestingly, in all tissue specimens of psoriatic skin examined (4/4), the 6F10 mAb also reacted with dermal cells of dendritic morphology distributed abundantly underneath the epidermis and extending processes to both blood vessels and the epidermal basement membrane (FIG. 8C, right panel). This cell type was not seen in normal human skin. To further characterize the LEEP-CAM-expressing dermal dendritic cells in psoriatic skin, double labeling was performed using the 6F10 mAb and mAbs against CD1a, CD14, and MHC class II. It was found that those dendritic cells in psoriatic skin co-expressed LEEP-CAM and MHC class II, but not CD1a or CD14. While the localization of these LEEP-CAM expressing dendritic cells was consistent with that of the recently described “lining macrophages” in psoriasis (Boehncke, W. H., et al. (1995) Am. J. Dermatopathol. 17:139-144), the lacking expression of CD1a suggested that this may be an as yet undescribed cell type in psoriatic skin, which may, as suggested by its expression of LEEP-CAM, interact with infiltrating T cells.

LEEP-CAM Mediates Adhesion of Activated T Cells to Normal and Psoriatic Epidermis

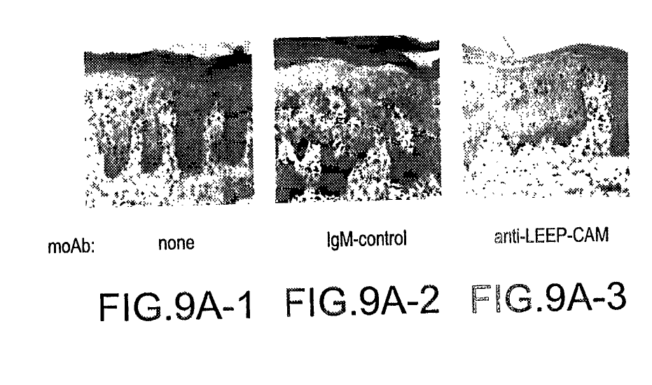

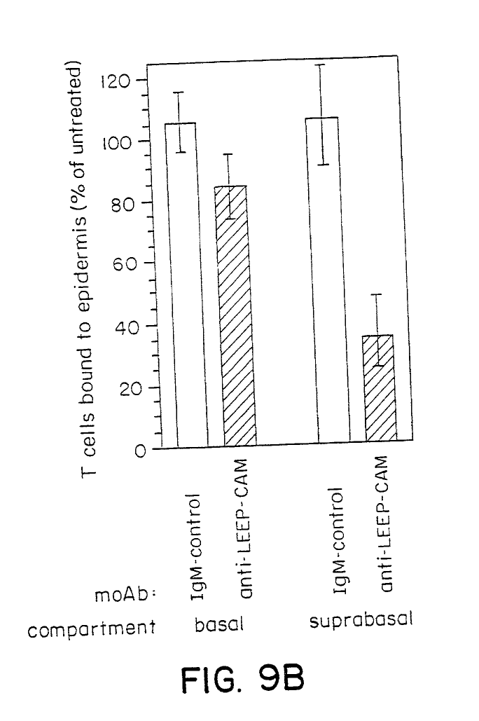

To directly assess the role of LEEP-CAM in adhesive interactions of T cells and components of inflamed and normal human skin, modified Stamper-Woodruff assays were performed. In these experiments, PHA-blasts (greater 92% activated CD3⁺ T cells as determined by flow cytometry) were allowed to adhere to cryostat-cut sections of normal or psoriatic human skin preincubated with the 6F10 mAb, an isotype-matched control mAb (N-S.4.1), the surface-binding BTI5 control mAb (Schön, M. P., et al. (1995) J. Invest. Dermatol. 105:418-42 5), or buffer only. In control sections, PHA-blasts homogeneously bound to the dermis as well as to basal and viable suprabasal layers of the epidermis, but not to the subcutaneous fatty tissue or to the stratum corneum indicating good specificity of the method (FIG. 9A). T cell binding to the dermal compartment was not affected in sections pre-incubated with the 6F10 mAb, as compared to the sections incubated with the control mAbs or buffer only (FIG. 9A). While the number of PHA-blasts bound to the basal layer of the epidermis (where LEEP-CAM is not expressed) was not altered significantly by incubation of the sections with the 6F10 mAb, binding to the suprabasal epidermal layers was reduced significantly by 67.2% comparing binding to sections incubated with control mAb and the 6F10 mAb (147.4 (SD=30.6) vs. 45.1 (SD=10.8) cells/mm skin, p=0.02, based on three independent experiments (FIG. 9B).

The overall binding of PHA-blasts to normal skin was markedly lower than that seen with psoriatic skin (14.6 (SD=2.2) cells/mm bound to the basal layer and 19.3 (SD=3.0) cells/mm bound to the suprabasal layers). Nevertheless, the 6F10 mAb still significantly reduced binding to the suprabasal layers by 67.8% (6.2 (SD=0.7) cells/mm, p=0.0008), but not the basal layer (11.6 (SD=1.2) cells/mm).

T Cell Migration into Monolayers of Immortalized Human Keratinocytes Mediated by LEEP-CAM.

To assess a possible role of LEEP-CAM in T cell migration into keratinocyte-derived tissues, a more complex function requiring the exertion of traction forces, a dynamic assay using modified Boyden-chambers was established. For this purpose, HaCaT cells were seeded onto the undersurface of polycarbonate filters with 8 μm-pores. HaCaT cells were used because normal keratinocytes were unable to adhere and form confluent monolayers on the polycarbonate membranes. HaCaT cells are spontaneously immortalized human keratinocytes which have preserved many phenotypic traits of normal keratinocytes including the expression of differentiation markers and the formation of orderly structured multilayered epithelia when transplanted onto nude mice (Boukamp, P., et al. (1988) J. Cell Biol. 106:761-771). In addition, as assessed by both flow cytometry and immunocytochemistry, HaCaT cells expressed high levels of LEEP-CAM (mean fluorescence intensities ˜200). Confluency of the HaCaT cells on the undersurface of the filters was confirmed by hematoxylin staining of representative filters.

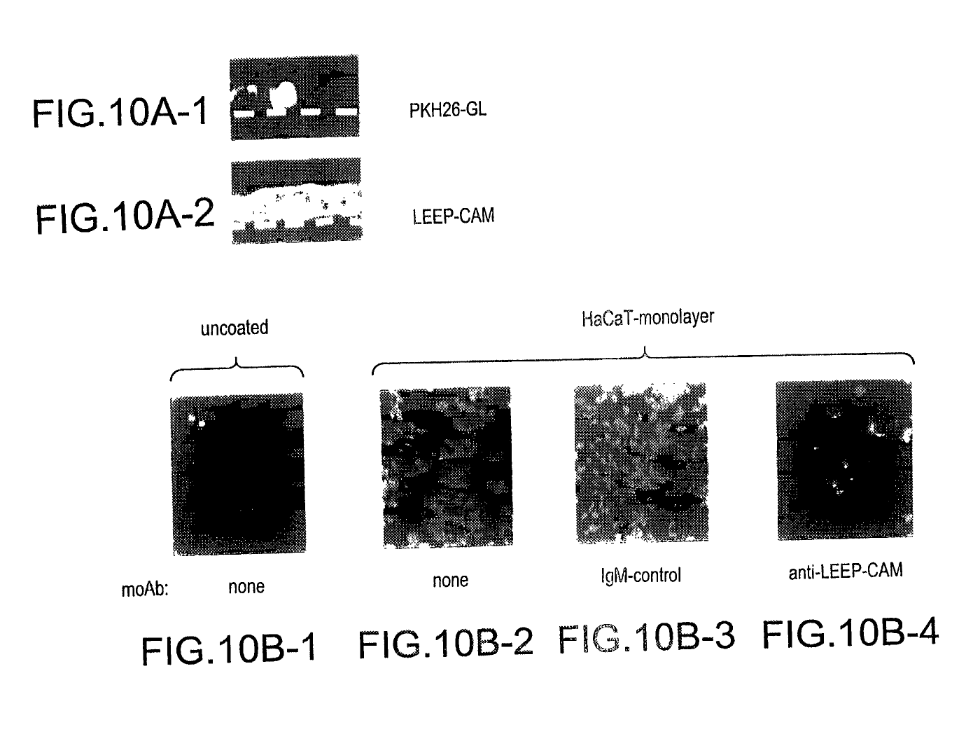

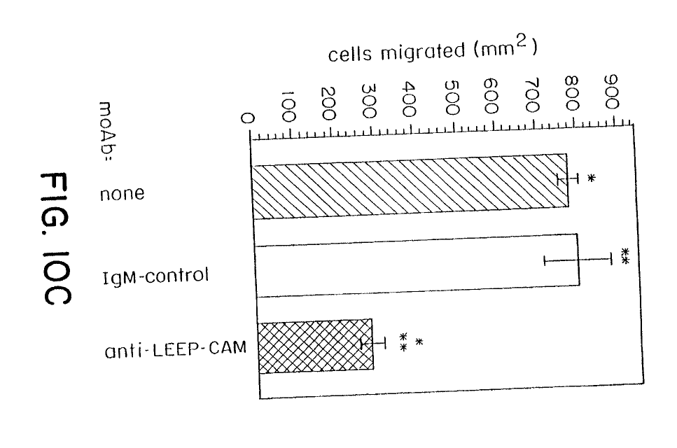

Activated T cells (PHA-blasts labeled with the intravital fluorescent dye PKH26-GL) were seeded into the upper compartment of the chambers and allowed to migrate into the HaCaT cell layer for 3.5 hours at 37° C. First, it was established by immunofluorescent staining of cryostat-cut sections of some representative filters that PHA-blasts migrated into the HaCaT-monolayer and did not adhere unspecifically to the filters (FIG. 10A). To examine the role of LEEP-CAM in this haptotactic migration process, filters were incubated prior to the migration assay with either no antibody, an isotype-matched IgM-control antibody, or the 6F10 mAb. After T cell migration, filters were mounted onto microscope slides and migrated cells were quantitated in a fluorescence microscope (FIGS. 10B and 10C). While a high number of activated T cells migrated into untreated (777, 4/mm², SD=26.0) or control treated HaCaT-monolayers (799.0/mm², SD=82.0), the number of migrated cells was reduced significantly by 63% in the 6F10-treated monolayers (289.1 /mm², SD=26.4, p=0.0001 and 0.003, respectively, FIG. 10C). In addition, T cells in the control chambers extended numerous processes into the HaCaT-layer, which was apparent by focusing up and down with the microscope, but cannot be visualized in two-dimensional figures. In contrast, T cells seeded onto 6F10-treated filters extended far less processes suggesting that blocking of LEEP-CAM efficiently inhibited interaction of activated T cells with cultured HaCaT cells.

LEEP-CAM Is Involved in Migration of Activated T Cells into Orthokeratinized and Stratified Organotypic Human Keratinocyte Cultures

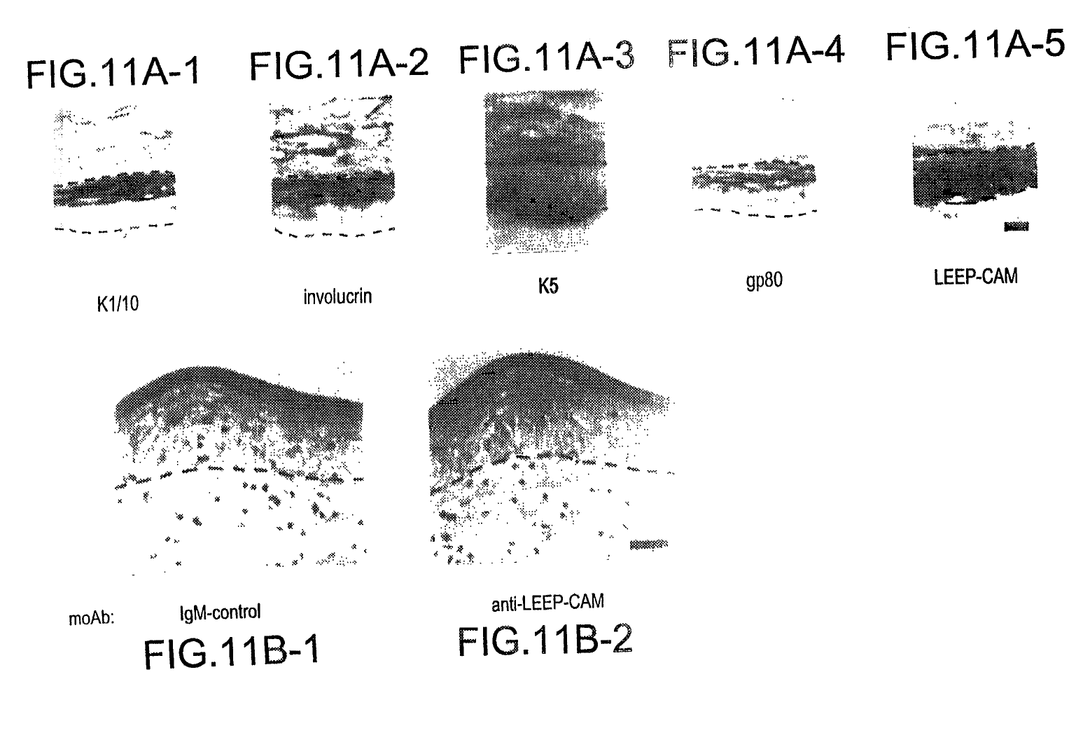

Although HaCaT-monolayer cultures used in the Boyden-chamber transmigration system provided important insights into the role of LEEP-CAM for the interaction of activated T cells with keratinocytes, these monolayers did not form stratified, orthokeratinizing, and polarized epithelia. As these epidermal differentiation characteristics may influence T cell migration and the spatial compartmentalization of infiltrating T cells, methods to overcome the limitations of a monolayer system were sought. To better approximate to the in vivo situation, organotypic cultures of normal human keratinocytes were generated (Schön, M., and J. G. Rheinwald (1996) J. Invest. Dermatol. 107:428-438.). These cultures were maintained on a collagen type 1/fibroblast dermis equivalent and cultured at the air/liquid interface to induce stratification and orthokeratinization. Similar to the in vivo situation, the organotypic cultures developed a well-defined basal layer of cuboidal cells, several viable suprabasal layers of flattening keratinocytes, and a well-developed cornified layer. The artificial epidermis of these cultures expressed keratins 1/10, involucrin, and gp80 in suprabasal viable layers. In addition, basal keratinocytes expressed keratin K5 (FIG. 11A). LEEP-CAM was expressed throughout the viable epidermal layers, but not in the cornified layer or in the dermis equivalent (FIG. 11A). To assess the usefulness of the organotypic cultures for functional experiments studying T cell interactions, modified Stamper-Woodruff assays were performed using cryostat-cut sections of organotypic cultures and activated T cells (PHA-blasts). In all sections, T cells strongly adhered to the dermis equivalent. In addition, in sections treated with the isotypematched control antibody N-S.4.1 or medium alone, T cells also bound to the viable epidermal layers (27.0 (SD=3.6) and 28.9 (SD=1.8) cells/mm epidermis, respectively), but not to the stratum corneum or the glass slide (FIG. 11B). In contrast, in sections incubated with the 6F10 mAb, T cell binding to the viable epidermal layers was reduced significantly by 42-46% (15.7 cells/mm (SD=1.7); p=0.04 and p=0.02, respectively), while binding to the dermis equivalents was not affected (FIG. 11B).

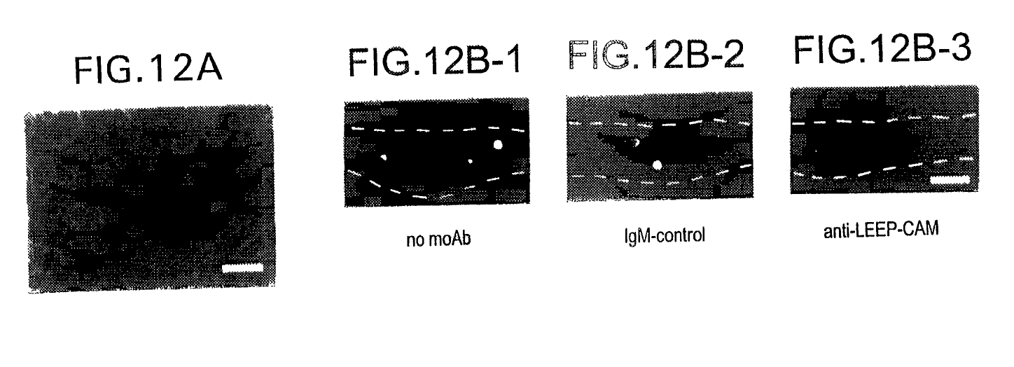

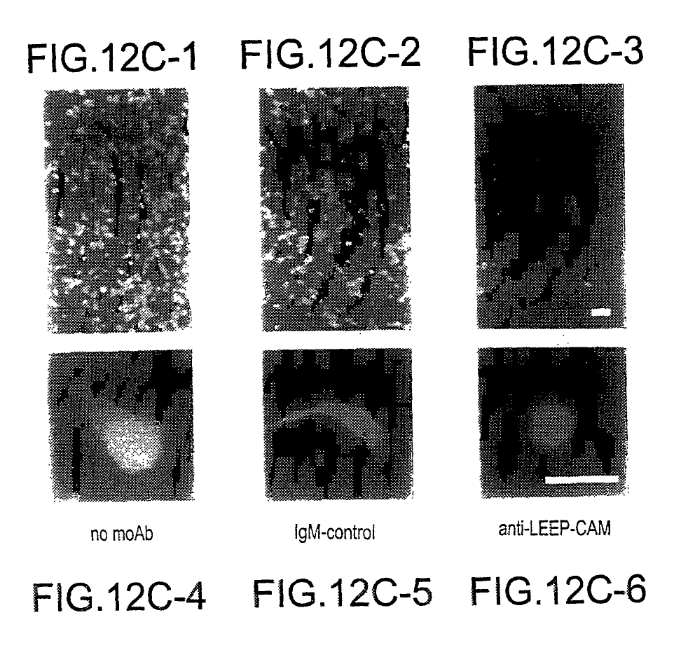

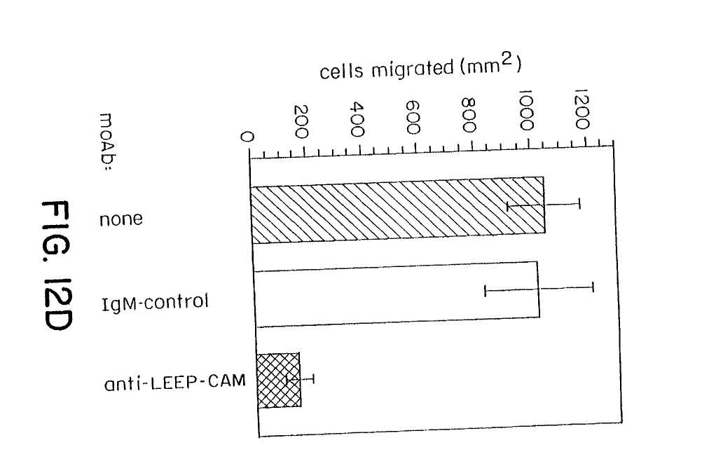

After removing the dermis equivalents and assessing penetration of the 6F10 mAb into the epidermal sheets (FIG. 12A), organotypic cultures were used for T cell migration assays as outlined in the Exemplification. Using cryostat-cut sections of organotypic cultures after a 3.5 h migration period, it was established that activated T cells abundantly migrated into the epidermal organoids. Indeed, T cell migration was seen into all viable epidermal layers, but not into the cornified layer, where LEEP-CAM was not expressed (FIG. 12B). When T cells were quantitated after migration into the organotypic keratinocyte cultures, it was found that high numbers of activated T cells migrated into untreated cultures (1041.7 cells/mm², SD=127.6), and into cultures treated with the two control mAbs N-S.4.1 or BT15 (1017.1 cells/mm², SD=192.2). In contrast, the number of migrated T cells was reduced dramatically by 85% in organotypic cultures treated with the 6F10 mAb (154.2 cells/cm², SD=52.5, p<0.00001, FIG. 12C). These results indicated that LEEP-CAM also mediated migration of T cells into well differentiated, polarized and stratified epidermal tissues in vitro. Indeed, the inhibitory effect of the 6F10 mAb appeared to be even more dramatic in organotypic cultures than in HaCaT-monolayer cultures or Stamper-Woodruff adhesion assays.

To confirm that this functional role of LEEP-CAM was a general mechanism rather than specific for PHA-blasts, the migration experiments into organotypic cultures were repeated using the TSBR-1 T cell line derived from skin lesions of atopic dermatitis, a common T cell-mediated skin disorder (Rossiter, H., F., et al. (1994) Eur. J. Immunol. 24:205-210). Again, TSBR-1 cells abundantly migrated into untreated organotypic cultures (444.2 cells/mm², SD=110.6) or into cultures pre-treated with either of the control mAbs N-S.4.1 or BT15 (431.7 cells/mm², SD=53.2 or 405.8 cells/cm², SD=85.1, respectively), while T cell migration was reduced significantly by greater than 90% in cultures pre-incubated with the 6F10 mAb (10.8 cells/cm², SD=6.6) as compared to the control cultures (p<0.0002).

Generation of Additional LEEP-CAM Specific Monoclonal Antibodies.

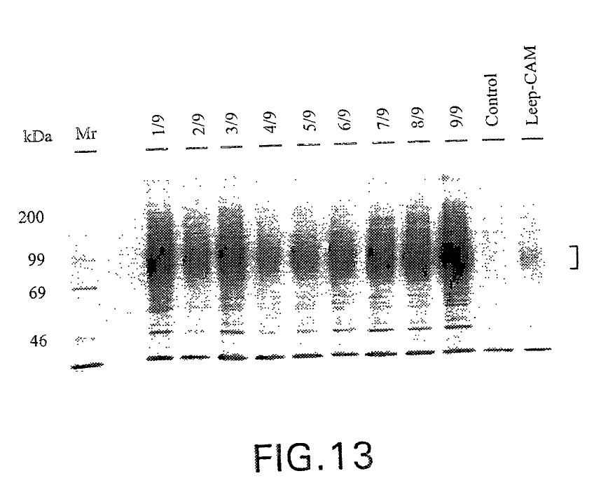

In addition to 6F10, nine mAbs, designated 1/9 to 9/9 have been generated by the method exemplified in Example 25. Like the 6F10 mAb, the newly generated antibodies recognize glycoproteins having a relative mobility of 70 kDa and 100 kDa from 16E6.A5 epithelial cells (FIG. 13) and block adhesion of IELs to epithelial cells in a static cell-cell adhesion assay. In addition, they were of the same isotype as the 6F10 mAb and recognize carbohydrate-dependent epitopes on LEEP-CAM.

Applications.

This invention describes a novel mechanism for tissue-specific localization of T cells to the human epidermis, a process crucial for immune surveillance and pathogenesis of cutaneous inflammation. LEEP-CAM (Lymphocyte Endothelial EPithelial-Cell Adhesion Molecule) was shown to mediate T cell migration into polarized, orthokeratinizing, multilayered and stratified epithelia expressing typical differentiation markers and exhibiting an orthokeratinizing differentiation pattern, thereby resembling normal human epidermis. Thus, LEEP-CAM is critically involved in a complex process requiring the exertion of traction forces by T cells as well as transient adhesive interactions between T cells and resident keratinocytes. As T cells must detach after binding to keratinocytes in lower epidermal layers in order to migrate into higher suprabasal epidermal layers, the LEEP-CAM mediated T cell-keratinocyte interaction appears to be regulated on the cellular level. It is likely that functional states of LEEP-CAM are altered during epidermal T cell localization. Switches between functional states due to conformational changes have been demonstrated for some integrin adhesion molecules (Springer, T. A. (1994) Cell 76:301-314; Hynes, R. O. (1992) Cell 69:11-2 5) and it is likely that LEEP-CAM is regulated similarly. In modified Stamper-Woodruff-assays, the LEEP-CAM mediated adhesion of activated T cells was markedly stronger to psoriatic as compared to normal epidermis, although LEEP-CAM is expressed in both. This indicates that different states of activation of LEEP-CAM exist and that activation of LEEP-CAM is upregulated in inflammatory conditions, e.g., by cytokines. Although proinflammatory cytokines, e.g., TNFα, IL-1 and IFNγ did not significantly alter the level of LEEP-CAM expression in cultured cells, functional states of LEEP-CAM could be regulated by cytokines.

Other mechanisms for epidermal localization of T cells, such as T cell binding to epidermal ligands through β1 integrins, ICAM-1/LFA-1 interactions, or binding to E-cadherin through the α^{E}β7 integrin expressed by some T cells, remain largely hypothetical. Most known ligands for T cell adhesion molecules, such as components of the extracellular matrix or VCAM-1, are not expressed beyond the epidermal basement membrane, suggesting that β1 integrins do not play a primary role in T cell epidermotropism, as was proposed previously (Sterry, W., et al. (1992) Am. J. Pathol. 141:855-860.). In contrast, LEEP-CAM is a T cell ligand expressed throughout all viable suprabasal epidermal layers, indicating that it is an important molecule in epidermal immune responses.

Induced by proinflammatory cytokines, there is de novo expression of epidermal ICAM-1 in some inflammatory conditions (Griffiths, C. E. M., et al. (1989) J. Am. Acad. Dermatol., 20:617-629; Dustin, M. L., et al. (1988) J. Exp. Med., 167:1323-1340; Groves, R. W., et al. (1992) J. Invest. Dermatol., 98:384-387; Kashihara-Sawami, M., and D. A. Norris. (1992) J. Invest. Dermatol., 98:852-856). Expression of ICAM-1 by keratinocytes and LFA-1 by T cells may mediate binding of activated T cells to inflamed epidermis (Shiohara, T., et al. (1989) J. Invest. Dermatol., 93:804-808). However, there also is evidence against this hypothesis. First, constitutive epidermal expression of ICAM-1 in transgenic mice does not lead to cutaneous T cell infiltration (Williams, I. R., and T. S. Kupper. (1994) Proc. Natl. Acad. Sci. USA, 91:9710-45). Second, there is no correlation of ICAM-1 expression by epidermal keratinocytes and LFA-1 expression by infiltrating T cells in canine mycosis fungoides (Olivry, T., et al. (1995) Arch.Dermatol. Res., 287:186-192). Third, ICAM-1 is expressed only focally in inflammatory skin conditions, and intraepidermal T cells frequently reside between ICAM-1-negative keratinocytes (Griffiths, C. E. M., et al. (1989) J. Am Acad. Dermatol., 20:617-629; Konter, U., et al. (1989) Arch. Dermatol. Res., 281:454-462; Kellner, I., et al. (1992) Br. J. Dermatol., 125:211-215). In contrast, suprabasal epidermotropic T cells reside between LEEP-CAM positive keratinocytes, and activated T cells do not migrate beyond the LEEP-CAM expressing layers in organotypic cultures.

Binding of integrin α^{E}β7 expressed by some T cell lymphomas to epidermal E-cadherin has been suggested to be involved in epidermotropism, similar to the mechanism proposed for intestinal epithelial T cell localization (Cepek, K. L., et al. (1994) Nature, 372:190-193). In vitro studies have demonstrated that α^{E}β7 binds E-cadherin (Cepek, K. L., et al. (1994) Nature, 372:190-193; Karecia, P. I., et al. (1995) Eur. J. Immunol., 25:852-856), and in vivo, α^{E}β7 is thought to mediate T cell localization to the intestinal mucosa (Parker, C. M., et al. (1992) Proc. Natl. Acad. Sci. USA, 89:924-1929). Given that some cutaneous T cell lymphomas expressed α^{E}β7, it was hypothesized that it mediates T cell epidermotropism in these cases (Sperling, M., et al. (1989) Am. J. Pathol., 134:955-960; Simonitsch, I., et al. (1994) Am. J. Pathol., 145:1148-1158). However, there are several lines of evidence, that the α^{E}β7-cadherin interaction may not be the primary mechanism for T cell epidermotropism. First, the majority of cutaneous T cell lymphomas does not express α^{E}β7 (Sperling, M., et al. (1989) Am. J. Pathol., 134:955-960). Second, its expression has not been reported in benign skin disorders exhibiting T cell epidermotropism. Finally, α^{E}β7 expressing lymphoma cells preferentially resided within the basal layer of the epidermis, whereas α^{E}β7 negative T cells were found in both basal and suprabasal layers (Sperling, M., et al. (1989) Am. J. Pathol., 134:955-960). It is also possible that expression of α^{E}β7 by cutaneous T cells “retains” those cells within the basal layer and actually hinders their migration into suprabasal layers. It seems, therefore, that mechanisms distinct from the α^{E}β7/E-cadherin interaction, such as T cell binding to LEEP-CAM, contribute to T cell epidermotropism in general.

As LEEP-CAM is expressed constitutively in normal uninflamed epidermis, it is possible that LEEP-CAM exerts another function distinct from T cell/keratinocyte adhesion. Such an alternative function could be homotypic adhesion between keratinocytes or adhesion between keratinocytes and other resident epidermal cells such as melanocytes, Merkel cells, or Langerhans cells. A similar dual function has been demonstrated for E-cadherin, which was initially identified as a homotypic and homophilic cell-to-cell adhesion molecule of epithelial cells involved in organ development during embryogenesis as well as tissue integrity within adult tissues (Takeichi, M. (1990) Annu. Rev. Biochem. 59:237-252. Later, it was shown that E-cadherin also mediates heterotypic and heterophilic adhesion between epithelial cells and the α^{E}β7 integrin expressed by some T cells (Kellner, I., et al. (1992) Br. J. Dermatol., 125:211-215; Cepek, K. L., et al (1994) Nature, 372:190-193; Karecia, P. I., et al. (1995) Eur. J. Immunol., 25:852-856).

Overall, LEEP-CAM mediates a novel mechanism for epidermal localization of T cells in inflammatory skin conditions. Given the importance of selective therapeutic strategies to treat inflammatory conditions without severe systemic side effects seen with general immunosuppressants, agents inhibiting the T cell epidermotropism mediated by LEEP-CAM can lead to selective alleviation of skin inflammation.

Thus, this invention relates to substances or compounds which are suitable for diagnosing or treating a condition involving a LEEP-CAM mediated inflammatory disease or disorder. Conditions or disorders which can be diagnosed or treated include, but are not limited to, arthritis, especially, Rheumatoid arthritis, asthma, Graft vs. Host disease, local infections, T cell-derived tumors (e.g., cutaneous lymphomas), dermatoses, inflammatory bowel diseases, autoimmune diseases, psoriasis, atopic eczema, lichen ruber planus, Crohn's disease, and ulcerative colitis.

In one embodiment, this invention is directed to a method of lessening or treating inflammation, in a mammal, especially a human, in vivo. The method comprises the steps of administering to a human or animal patient in need of such a treatment, efficacious levels of a LEEP-CAM binding compound which prevents binding of T or B cells to the 6F10 antigen. By “efficacious”, it is meant that the amount administered is at a sufficient level to ameliorate or prevent inflammation due to LEEP-CAM adhesion-mediated T or B cell migration into the tissues beyond the normal migratory state during periods when the subject is not suffering an inflammatory reaction. In a particularly useful embodiment the area of inflammation to be treated can be selected from distribution in suprabasal region of the epidermis, the basal layer of bronchial epithelia, the basal layer of breast epithelia, the tonsillar epithelia, the vaginal epithelia, the vascular epithelium, or the high endothelial venule endothelia.

The LEEP-CAM antagonist can be administered on a regular basis in low doses to prevent the onset of inflammatory disorders. Alternatively, efficacious doses of the reagent can be utilized as a treatment during the course of an inflammation to prevent further lymphocyte trafficking or influx into the affected tissues or organs, so that further inflammation can be avoided.

Further methods of treating a mammal to decrease or prevent an inflammatory response can comprise identifying an area of the mammal having a local inflammatory response and administering a therapeutic composition comprising a LEEP-CAM inhibitor in a therapeutically effective amount to the area of local inflammatory response, whereby LEEP-CAM molecules are unable to interact with lymphocytes in the area of local inflammatory response, whereby the inflammatory response is decreased. These methods would be especially useful in bodily areas of mammals such as the suprabasal region of the epidermis, the basal layer of bronchial epithelia, the basal layer of breast epithelia, the tonsillar epithelia, the vaginal epithelia, the vascular epithelium, and the high endothelial venule endotelia. Thus, either T or B lymphocyte activity can be suppressed through modulation of LEEP-CAM binding to or mediated migration of these lymphocytes.

In another embodiment, LEEP-CAM activity can be upregulated to increase the influx of T or B cells into a particular tissue, thus increasing the inflammatory response. By “upregulation” it is meant that LEEP-CAM mediated lymphocyte migration is increased because the amount of LEEP-CAM and/or its expression in a particular tissue is increased. Upregulation can be accomplished by several methods, depending on the means by which LEEP-CAM activity is maintained at normal levels or is reduced in the tissue in which the upregulation is to occur. One method, without limitation to this example, could be the use of a therapeutic composition, such as a small molecule which increases expression of LEEP-CAM where it is present but maintained at low levels. Another means could encompass increasing the amount of LEEP-CAM in a particular tissue. In either of these examples, migration of T or B cells can be increased to produce an inflammatory response. This could be useful, for example, where tumors occur and there is a loss of LEEP-CAM expression.

Suitable LEEP-CAM binding agents can include small molecules, especially compositions which preferentially bind to LEEP-CAM compared to other cellular adhesion molecules and which interfere with (downregulate) or upregulate LEEP-CAM mediated lymphocyte migration in LEEP-CAM positive tissues. Small molecules which affect LEEP-CAM and its activity, either through direct binding to LEEP-CAM or indirectly through other cellular activity) can be screened from a chemical library through an assay system. For example, given cells which are positive for the 6F10 antigen and cells which are negative for the presence of 6F10 antigen, an assay system can be designed wherein small molecules can be screened for their capabililty to affect 6F10 antigen expression and/or activity. These molecules can then be selected on the basis of efficacy in upregulating or downregulating LEEP-CAM mediated migration of lymphocytes.

Other LEEP-CAM binding agents include antibodies, preferably monoclonal antibodies such as 6F10 or antibody fragments. If antibodies are employed as antagonists, they can be prepared by any suitable technique. LEEP-CAM or any portion of the molecule can be used to induce the formation of anti-LEEP-CAM antibodies, which can be identified by routine screening. Alternatively, T or B cell ligands which bind to LEEP-CAM resulting in adhesion-mediated migration of the T or B cells can induce formation of antibodies. These antibodies can also be effective inhibitors of LEEP-CAM cell adhesion, thus preventing T or B cell trafficking into affected tissues.

In particular, an antibody of this invention, especially a monoclonal antibody, would bind to a 90-115 kDa or a 145 kDa cell surface glycoprotein which can modulate the migration of lymphocytes into epithelial layers of a mammal. Other properties of the antigen would include its capability to modulate lymphocyte adhesion and migration independent of the presence of cations.

Antibodies can either be polyclonal or monoclonal antibodies, or antigen binding fragments of such antibodies (e.g., F(ab) or F(ab)₂ fragments). Polyclonal antibodies generally are raised in animals by multiple subcutaneous or intraperitoneal injections of the appropriate antigen or mimitope, together with an adjuvant. Mimitopes are cross-reacting epitopes which are conformationally related to the antigen due to similarities in three dimensional folding rather than amino-acid sequence. Monoclonal antibodies are prepared by recovering immune cells, typically spleen cells or lymphocytes from lymph node tissue, from animals immunized with the appropriate antigen and immortalizing the cells in conventional fashion, e.g., by fusion with myeloma cells or by Epstein-Barr virus transformation and screening for clones demonstrating expression the desired antibody. Human hybridomas can be used in these methods to produce human monoclonal antibodies. Standard methods for the production of these antibodies and methods for their purification can be found in, e.g., Harlow, E. and D. Lane (1988) Antibodies: A Laboratory Manual, Cold Spring Harbor Laboratory Press, Cold Spring Harbor, N.Y.; Ausubel et al. (1994) Current Protocols in Molecular Biology, Vol. 2, Chapter 11 (Suppl. 27) John Wiley & Sons: New York, N.Y.).

Techniques for creating recombinant DNA versions of the antigen-binding regions of the antibody molecules (known as Fab fragments), which bypass the generation of monoclonal antibodies, are encompassed withing the practice of this invention. Antibody-specific mRNA from immune system cells taken from an immunized animal is extracted, transcribed into complementary DNA (cDNA), and cloned into a bacterial expression system, an animal (including human) cell or a plant cell. The expressed Fab fragment can be harvested, transported to the periplastic space or secreted, if in a bacterial cell, or harvested by an appropriate procedure from other types of cells.

The term “treatment” or “treating” is intended to include the administration of a LEEP-CAM binding compound to a subject for purposes which can include prophylaxis, amelioration, prevention or cure of disorders mediated by LEEP-CAM adhesion to T lymphocytes. When administered to a human or animal, the reagents of this invention can be formulated in any manner which makes it suitable for cutaneous, parenteral or mucosal administration. The reagent can be in the form of, for example, an injectable solution, aerosol formulation, suspension, topical formulation, enema, etc. For example, an anti-LEEP-CAM agent can be contained in a transdermal patch for treatment of psoriasis or other dermatological condition. In another example, reagents for treatment of asthma can be in the form of a nasal spray or produced in an inhaler.

These agents can be formulated with pharmaceutically-acceptable excipients or carriers, such as isotonic saline, in accordance with conventional pharmaceutical practice. The dosage level of the reagent will be sufficient to provide an anti-inflammatory effect by blocking LEEP-CAM mediated migration of T cells. The reagent can be conjugated to other compounds for the purpose of enhancing or provided additional properties which enhance the reagent's ability to provide relief of LEEP-CAM mediated effects.

The amount and regimen for the administration of inhibitors of LEEP-CAM mediated T or B cell adhesion and migration can be determined readily by those of ordinary skill in the clinical art of treating inflammation-related disorders such as psoriasis and tissue injury. In general, dosages will vary depending on considerations such as: type of reagent employed, age, health, gender, medical condition, concurrent treatments, if any, frequency of treatment, nature of the effect sought, duration of the symptoms, counterindications, if any, and other variables. The dosage can be administered in one or more applications to obtain the desired results, or as a sustained-release form.

This invention also relates to diagnostic methods and reagents for the detection of LEEP-CAM protein and LEEP-CAM binding of lymphocytes in cells of mammals, especially humans, to assess a medical condition. These methods can thus be used to detect skin diseases, such as psoriasis and other inflammatory disorders.

The methods can comprise detecting anti-LEEP-CAM antibody binding to LEEP-CAM positive cells taken in a sample from a subject (such as a skin biopsy), and diagnosing the medical condition on the basis of such binding. In an alternative embodiment, an antibody which binds to a mimitope of LEEP-CAM can be substituted for the anti-LEEP-CAM antibody when diagnosing the medical condition. Diagnostic methods using antibodies in vivo can also be used.

Examples of such reagents are LEEP-CAM binding compounds, including an antibody, preferably a monoclonal antibody or an antibody fragment with specificity for a LEEP-CAM epitope, such as 6F10 or mAbs 1-9/9. The antibody can be labeled with a substance which permits the detection of binding of the antibody to the isolated LEEP-CAM or to cells which express LEEP-CAM on their surface. Such diagnostic compositions can be provided in a kit. An example would be,

a) an antibody, preferably a monoclonal antibody, with specificity for LEEP-CAM, or a biologically active derivative of the antibody, preferably labeled with a substance which permits detection of binding of the antibody to LEEP-CAM; and

b) purified LEEP-CAM, to provide a standard for evaluation of the assay results.

### EXEMPLIFICATION

### Example 1

Cells and Cell Culture

The breast epithelial cell 16E6.A5 (Dr. V. Band, Tufts University, New England Medical Center, Boston, Mass.) was derived by immortalization of the 76N normal human mammary epithelial cell line through transfection of the E6 and E7 genes of the human papilloma virus (Band, V. and Sager, R. (1989) Proc. Natl. Acad. Sci. USA 86:1249-1253; Band et al., (1990) Proc. Natl. Acad. Sci. USA 87:463-467). The clone was grown in DFCl-1 medium that consists of a-MEM/HAM nutrient mixture F12 (1:1, vol./vol.) (Gibco, Grand Island, N.Y.) supplemented with 12.5 ng/ml epidermal growth factor, 10 nM triiodothyronine 10 mM Hepes, 50 μM freshly dissolved ascorbic acid, 1 nM γ-estradiol, 1 μg/ml insulin, 2.8 μM hydrocortisone, 0.1 mM ethanolamine, 0.1 mM phosphoethanolamine 10 μg/ml transferrin, 2 mM L-glutamine, 100 U/ml penicillin and 100 μg/ml streptomycin sulfate, 15 nM sodium selenite (all from Sigma Chemical Co. St. Louis, Mo.), 1 ng/ml cholera toxin (Schwatz/Mann, New York) and 1% fetal calf serum (FCS, Hyclone Laboratories, Logan, Utah).

Human umbilical vein endothelial (HUVEC) cells (Jaffe et al., (1973) J. Clin. Invest. 52:2745-2756) were maintained in culture under standard conditions on 1% gelatin coated flasks with 199 media (Gibco) supplemented with 20% FCS, 90 μg/ml heparin (Sigma) and 20 μg/ml endothelial growth supplement (EGS) (Sigma). HUVEC passed 5-10 times were used for adhesion assays in this study.

The CDC/EU.HMEC-1 (HMEC-1) endothelial cell line (Bosse et al., (1993) Pathobiology 61:236-238) was derived from microvascular endothelial cells from human foreskin and was grown in endothelial basal media (Clonetics, San Diego, Calif.) supplemented with 2 mM L-glutamine, 12.5 ng/ml epidermal growth factor, 2.8 μM hydrocortisone, 100U/ml penicillin, 100 μg/ml streptomycin sulfate, and 5% FCS.

ECV304 is a spontaneously transformed endothelial cell line derived from a human umbilical cord (Takahashi et al., (1990) In Vitro Cellular & Developmental Biology 26:265-274) and was grown in 199 media with 10% FCS, available from ECACC.

Peripheral blood mononuclear cells (PBMC) were isolated from heparinized human whole blood by density separation over Ficoll-Hypaque (Pharmacia Chemicals, Uppsala, Sweden). Monocytes were separated from PBMC by incubating the PBMC in plastic tissue culture flasks for 1 hour. The adherent cells were collected as blood monocytes. The polymorphonuclear leukocytes (PMN) were isolated from the peripheral blood by diluting 1:1 with ACD (4.5 ml acid citrate: 6 ml dextran) and allowed to settle for one hour.

Leukocyte rich plasma overlaying the settled red blood cells was then separated by Ficoll-Hypaque centrifugation and the pellets were collected, the remaining RBCs lysed with hypotonic saline and the remaining leukocytes were washed with PBS and suspended in adhesion medium and used in adhesion assays.

The human intestinal intraepithelial lymphocyte (iIEL) cell line 3901 was derived from intestinal epithelium as previously described (Russell et al., (1994) Eur. J. Immunol. 24:28322-2841). The iIEL line was cultured in Yssel's medium (Yssel et al., (1984) J. Immunol. Methods 72:219-227) containing 2 nM rlL-2 (Ajinomoto, Kawasaki, Japan), 4% (v/v) FCS (HyClone), and 50 μM 2-ME at 10% CO₂. Long term culture of the 3901 ilEL line was maintained by intermittent restimulation with phytohemagglutinin-P (PHA; Difco, 1:2000) and irradiated feeder cells (80% PBMC and 20% JY lymphoblastoid cells).

PHA blasts were derived by stimulating PBMC or CD4+ or CD8+ subpopulations of PBMC with PHA (Difco, 1:2000) and irradiated feeder cells (JY lymphoblastoid cells) in Yssel's medium containing 2 nM recombinant interleukin (IL)-2 (Ajinomoto), 4% (vol/vol) fetal calf serum (Hyclone), and 50 μM2-mercaptoethanol and grown in 10% CO₂.

The T84 colon human carcinoma cell line was obtained from ATCC and grown in DMEM/HAM nutrient mixture F12 (1:1, vol/vol)(Gibco) supplemented with 15 mM HEPES, 1.2 g/liter NaHCO₃, 40 mg/liter penicillin, 8 mg/liter ampicillin, 90 mg/liter streptomycin sulfate, and 5% (vol./vol.) fetal calf serum (Hyclone). Confluent monolayers of T84 cells were subcultured by incubation with a 0.1% trypsin/0.9 mM EDTA solution in phosphate buffered saline for 20 min. at 37° C.

A431 (epidermal carcinoma cell line), 293T cells (transformed embryonic kidney cell line), ThP1 (monocytic cell line), U937 (histiocytic lymphoma), HL60 (premyelocytic leukemia), and JY cells (B cell leukemia) are human permanent cells lines available from American Type Culture Collection (ATCC, Rockville, Md.) and were cultured in RPMI-1540 media containing 5% FCS.

To generate activated T cells, peripheral blood lymphocytes (PBL) were isolated by density gradient centrifugation using Ficoll (GibcoBRL, Grand Island, N.Y.) and cultured in RPMI 1640 supplemented with 10% fetal calf serum (FCS), 0.3% Phytohemagglutinin (PHA), 15 mM HEPES, 2 mM L-glutamine, and 100 U/ml penicillin/streptomycin (all from GibcoBRL). Cells were used for functional experiments after 1 to 2 weeks. TSBR-1 is a human T cell clone derived from skin lesions of atopic dermatitis (Rossiter, H., F. et al. (1994). Eur. J. Immunol. 24:205-210). These cells were cultured in RPMI 1640 supplemented with 10% FCS, 2 mM L-glutamine, 100 U/ml penicillin/streptomycin, 15 mM HEPES, and 2 ng/ml IL-2. HaCaT cells are spontaneously immortalized human keratinocytes (Boukamp, P., et al. (1988) J. Cell Biol. 106:761-771.) and were maintained in DMEM supplemented with 10% FCS, 100 U/ml penicillin/streptomycin, and 2 mM L-glutamine.

Organotypic cultures using the normal human keratinocyte strain N and the dermal fibroblast strain B03 8 (Lindberg, K., and J. G. Rheinwald (1990) Differentiation45:230-241.) were prepared as described in Schön, M., and J. G. Rheinwald (1996) J. Invest. Dermatol. 107:428-438, with minor modifications. Briefly, 1 ml of an acellular solution containing 0.7 mg/ml of bovine type I collagen (Organogenesis, Canton, Mass.) was cast on six-well tissue culture tray inserts equipped with a polycarbonate membrane with 3-μm-pores. Three ml of collagen solution containing 2.3×10⁴ B038-fibroblasts per ml then were cast on top of this layer. The embedded fibroblasts were allowed to contract and reorganize the collagen matrix during a 4-day incubation period at 37° C. and 5% CO₂. Human keratinocytes then were seeded on top of these collagen/fibroblast dermis equivalents at 2×10⁵ cells/cm². The cultures then were maintained for four days submerged in DMEM/F12 (3:1 v:v) supplemented with 0.3% bovine serum, 5 μg/ml insulin, 0.4 μ/ml hydrocortisone, 20 pM trilodthyronine, 5 μm/ml transferrin, 10⁴ M ethanolamine, 10⁴ M phosphoethanolamine, 5.3×10 ⁸ M selenious acid, and 1.8×10⁻⁴ M adenine. Cultures then were raised to the air-liquid interface to induce stratification and keratinization. The development of stratified epithelia was monitored by histology using representative cultures and the organoids were used for functional experiments after 10 days at the air-liquid interface.

### Example 2

Magnetic Cell Sorting

CD4+ and CD8+ Lymphocytes were purified from PBMC with Magnetic Cell Sorting (Miltenyi Biotech, Hamburg, Germany). Briefly, 10⁷ cells suspended in 80 μl PBS/5% FCS were incubated for 20 minutes in 20 μl anti-CD4/CD8 mAb coupled magnetic Biobeads (Miltenyi Biotech) for 15 minutes on ice. After washing once, cells were passed through a column with a strong magnetic field. After extensive washing, the column was removed from the magnetic field and the bound cells were eluted with 5 column volumes of PBS/5% FCS. The eluted cells were then subjected to flow cytometry analysis with the corresponding mAb. The purity of the cells was routinely more than 90%.

### Example 3

Monoclonal Antibodies

Monoclonal antibody (mAb) 6F10 (mouse Ig Mκ) was generated by immunizing BALB/cJ mice with the human breast cancer cell line 16E6.A5. Three intraperitoneal injections and a final intravenous injection of 2×10⁷ cells were given at 3 week intervals. Three days after the intravenous immunization, splenocytes were isolated and fused with P3X63Ag8.653 myeloma cells in the presence of PEG 1450 as described previously (Kohler, G. and Milstein, C. (1975) Nature 256:495-497; Barnstable et al., (1978) Cell 14:9-20; Hochstenbach et al., (1992) Proc. Natl. Acad. Sci. USA 89:4734-4738. Hybridomas were selected with aminopterin-containing medium, and hybridoma supernatants were screened by adhesion assays to detect blocking of adhesion of ilEL to epithelial cell monolayers. The selected hybridomas were subcloned three times by limiting dilution, and ascites containing the antibody was produced by intraperitoneal injection of the hybridoma cells into pristane-treated BALB/cJ mice. The isotype of this antibody is IgMκ, determined with an ELISA isotyping method (Amersham). The isotype of this antibody, MAbN-S.4.1 (nonbinding mouse IgMκ), was obtained from the ATCC and was used as control.

Previously described mAb used were NS4.1 (mouse anti-sheep RBC, IgM), BerACT8 (mouse anti-human α^{E}β₇, lgG1) (Kruschwitz et al., (1991) J. Clin. Pathol. 44:636-645, E4.6 (mouse anti-human E-cadherin, IgG1) (Cepek et al., (1994) Nature 372:190-193, TS 1/22 (mouse anti-human LFA-1, lgG1), (Sanchez-Madrid et al, (1982) Proc. Natl. Acad. Sci. USA 79:7489-7493, 4B4 (mouse anti-human β₁, lgG1) (Morimoto et al., 1985) W6/32 (mouse anti-human MHC class 1, IgG2a) (Barnstable et al., (1978) Cell 14:9-20), OKT3 (mouse anti-human CD3, IgG2a), available from American Type Culture Collection (ATCC).

The BT15 MAb (mouse IgG1) binds to an 80 kDa cell surface glycoprotein (gp80) that is expressed in suprabasal human keratinocytes committed to terminal differentiation (Schön, M. P., et al, (1995) J. Invest. Dermatol. 105:418-425; Schön, M. P., et al., (1995) Arch. Dermatol. Res. 287:591-598). The epidermal distribution pattern in vivo and the expression by cultured keratinocytes of this molecule are very similar to those of LEEP-CAM (Schön, M. P., et al., (1995) J. Invest. Dermatol. 105:418-425). As surface-binding mouse IgMκ-controls were not available, this mAb was used as a surface binding control. Monoclonal antibodies OKT6 (human CDla), c1322 and 3C10 (human CD 14), L243 (human MHC class II), P3 (IgG1-control) and GE2.9.5 (IgG2a-control), were used in two-color immunohistochemistry. MAbs AE2 (anti-human keratin K1/10), 6B10 (anti-human keratin K4; Sigma), AE14 (anti-human keratin K5), AKH1 (anti-filaggrin), or IIA58 (anti-ICAM-1; Pharmingen, San Diego, Calif.) were used in immunohistochemistry. Hybridomas producing mAb were grown in RPMI1640 supplemented with 10% Ig-depleted fetal calf serum (FCS), 10⁻⁵ M 2-mercaptoethanol, 100 U/ml penicillin/streptomycin, 2 mM L-glutamine, and 15 mM HEPES-buffer. Mouse IgM was purified using protein G (Pharmacia, Uppsala, Sweden) covalently linked to rat-anti-mouse-κ-chain (mAb 187.1, ATCC), and mouse IgG was purified using protein G (Pharmacia). For all experiments, mAbs were used at 20 μg/ml or, alternatively, as 1:20 diluted ascites. For antibody biotinylation, 10 μl of NHS-LC-biotin (11.3 mg/ml; Pierce, Rockford, Ill.) were added to 500 μg of purified mAb. The solution then was incubated for 2 hours at room temperature in the dark, and dialyzed against PBS overnight.

### Example 4

cDNA Clones

A human ICAM-1 cDNA clone pCD1.8 was obtained from Cr. T. A. Springer (Diamond et al., (1991) Cell 65:961-971). Human cDNA clones of the CD44 isoforms, CD44H and CD44E, and the parental expression vector pCDM8 were obtained from Dr. B. Seed and Dr. I. Stamenkovic (Stamenkovic et al., (1991) Cell 56:1057-1062).

### Example 5

Cytokines

Recombinant IL-1 α and β were obtained from DuPont through the biological Response Modifier Program, National Cancer Institute, National Institute of Health (Bethesda, Md.). Recombinant TNF-α and IFN-γ 1b were obtained from Genentech Inc., (San Francisco, Calif.). 10 U/ml of each cytokine was used in cell culture stimulation experiments.

### Example 6

Adhesion Assays

Adhesion assays were performed as previously described (Cepek et al., (1993) J. Immunol. 150:3459-3470) with modifications. Briefly, monolayers of adherent cells were grown in 96-well flat bottom tissue culture plates. 10⁴ adherent cells were cultured in each well and allowed to grow to confluence. The monolayers were washed twice with PBS before the adhesion assay. In antibody blocking experiments, the adherent cells were incubated with 50 μl hybridoma culture supernatant, 1/250 dilution of ascites or 10 μg/ml of purified mAb for 30 minutes before adding the suspension cells. Suspension cells were labeled with 25 μg of 2′,7′-bis-(2-carboxyethyl)-5 (and -6) carboxyfluorescein (BCECF-AM, Molecular Probes, Inc. Eugene, Oreg.) dissolved in 5 μl of DMSO and added to complete culture media for 30 minutes in 37° C. After washing with PBS, 40,000 labeled suspension cells were resuspended in 100 μl of adhesion media (50 mM Tris-HCl, pH 7.4, 150 mM NaCl, 1 mM CaCl₂, and 2 mM MnCI₂) with or without blocking antibodies and added to each well of adherent cells and incubated at 37° C. for 50 minutes. Unbound cells were then washed from the plates with adhesion media (3 to 5 washes). Bound cells were detected using a fluorescence plate reader (IDEXX Co., Portland, Me.). The bound cells were read as fluorescence units shown on the reader. At least four replicates were performed in each experiment. If not specified, the bound cells routinely account for 20-40% of the input cells after 3-5 washes when epithelial cells (16E6.A5) were used as the adherent cells. Student's t test was used to analyze the data obtained in adhesion assays.

### Example 7

Flow Cytometry

Flow cytometry analysis was performed as previously described using the FACSort flow cytometer (Becton Dickinson, Mountain View, Calif.). Primary and secondary antibodies were used at saturating concentrations. Isotype matched irrelevant mAbs were used as negative controls while W6.32 antibody (mouse anti-human MHC class 1) was used as a positive control. The mean fluorescence intensity (MFI) of negative controls was consistently less than 10 fluorescence units.

### Example 8

Cell Surface Treatment with O-glycoprotease

O-sialoglycoprotease was obtained from Cedarlane (Homby, Ontario). Fifty μl of reconstituted enzyme was added to 1×10⁷ live cells suspended in 0.5 ml of RPMI-1640 medium. The samples, with or without enzyme, were incubated at 37° C. for 1 hour, washed twice with PBS and subjected to FACS analysis.

### Example 9

Lipofectamine Transfection

293T cells were cultured in 6 well plates in DME containing 10% FCS until the cells were about 50-70% confluent. For each well of cells, the following were prepared: a) 1μ of DNA in 100 μl of Opti-MEM (Gibco) and b) 10 μl of Lipofectamine (Gibco) in 100 μl of Opti-MEM. The two solutions were mixed and incubated at room temperature for 30 minutes. Before completion of the incubation, the cells were rinsed once with Opti-MEM. 0.8 ml of Opti-MEM was then added to the mixture, then the entire DNA-Lipofectamine mixture was added into the cell culture. The transfection was allowed to proceed for 5 hours at 37° C. and 10% CO₂, then 1 ml of DME with 20% FCS without antibiotics was added to each well. The cell culture media were changed to normal media after 24 hours. The cells were analyzed 48 hours after beginning the transfection.

### Example 10

Immunohistochemistry

Tissue samples were mounted in OCT compounded (Ames Co. Elkart, Ind.), frozen in liquid nitrogen and stored in −70° C. Frozen tissue sections, 4 μm thick, were fixed in acetone for 5 minutes, air dried, and stained by an indirect immunoperoxidase method (Cerf-Bensussan et al., (1983) J. Immunol. 130:2615-2622) using avidin-biotin-peroxidase complex (Vector Laboratories, Bulingame, Calif.) and 3-amino-9-ethylcabazole (Aldrich Chemical Co., Inc. Milwaukee, Wis.) as the chromogen.

### Example 11

SCID-human Skin Zenograft Model

Human neonatal foreskin was grafted onto the back of a 6-8 week old SCID mice and allowed to heal for 4 weeks (Kim et al., (1992) J. Invest. Dermatol 98:191-197). 5000 units of recombinant human TNF-α (Genentech) in 50 μl of sterile saline was injected into one site of the biopsy. The control site (on the same skin sample) was injected with 50 μl of sterile saline alone. 24 hours later, the mice were sacrificed and 5 mm circular punch biopsies were taken from the control and TNF-α injected sites. Sections were taken for immunochemical staining. Primary antibodies for immunohistochemical staining were diluted in PBS with 1% FCS and used as follows: E-selectin (R&D systems, 1 μg/ml), PECAM-1 (R&D systems, 1 μg/ml), and 6F10 (1:100 dilution from ascites).

### Example 12

Immunoprecipitation

Epithelial and endothelial cells were labeled with either Na¹²⁵I (Dupont-New England Nuclear) cell surface labeling or ³⁵S methionine and cysteine (DuPont-NEN) metabolic labeling as previously described (Brenner et al, (1987) J. Immunol. 138:1502-1509). The cells were solubilized in lysis buffer containing Tris buffered saline (TBS, pH 7.6) with 1% Triton-X-100, 0.5% sodium deoxycholate (DOC), 8 mM iodoacetamie and I mM phenylmethylsulfonyl fluoride (Sigma) for one hour. After centrifugation to remove insoluble debris, the lysates were precleared with 200 μl of Staphylococcus aureus Cowen strain I (Pansorbin, Calbiochem, La Jolla, Calif.). Lysates from 3-5×10⁵ cells were used in each immunoprecipitation. The lysates were incubated with either 100 μl of 10% (v/v) antibody coupled Sepharose 4B (Pharmacia Inc. Piscataway, N.H.) or 0.5 μl of ascites and 125 μl of culture supernatant of 187.1 hybridoma (mouse anti-human κ chain) followed by incubation with 100 μl of protein A-Sepharose (Pharmacia Inc. Piscataway, N.J.). The immunoprecipitates were washed five times with TBS with 1% Triton-X-100, 0.5% DOC, and 0.2% SDS, eluted with sample buffer containing 10% glycerol, 3% SDS, and 5% 2-ME by boiling for 3 minutes and resolved by 10.5% or 7.5% SDS-polyacryamide gel electrophoresis as described (Hochstenbach et al., (1988) J. Exp. Med. 168:761-776). For N-glycanase treatment of immunoprecipitate, washed beads were resuspended in 50 μl of 30 mM Tris buffer (pH 7.6), 0.1% SDS and 0.1M 2-ME. The samples were boiled for 5 minutes to denture the proteins. Five 1 μl of 10% TX100 was added to the elution after samples cooled to room temperature and 0.3 U N-glycanase (Genzyme, Boston) was added. The reaction was allowed to proceed overnight at 37° C.

### Example 13

Two Dimensional IEF/SDS-PAGE Analysis

Immunoprecipitates were dissolved in isoelectric focusing (IEF) sample buffer containing 9.33M urea, 2.5% Triton X-100, 5% 2-ME, and 2% ampholines (pH3.5-10; Pharmacia) and resolved by IEF in the first dimension in a slab gel. The first dimension gel was incubated in equilibration buffer (containing 23 mM Tris, pH6.8, 10% glylcerol, 2.5% SDS, and 5% 2-ME) then subjected to 7.5% SDS-PAGE in the second dimension under reducing as previously described (Brenner et al., (1987) J. Immunol. 138: 1502-1509).

### Example 14

Protein Purification and Amino Acid Sequence Analysis of the 6F10 Antigen

Forty grams of 16E6.A5 epithelial cells were solubilized in 40 ml lysis buffer containing Tris buffered saline (TBS, pH 7.6) with 1% Triton-X-100, 0.5% sodium deoxycholate (DOC), 8 mM iodoacetamide and 1 mM phenylmethylsulfonyl fluoride (Sigma Chemical Co., St. Louis, Mo.) for one hour. After centrifugation to remove insoluble debris, the lysates were passed through nonspecific column coupled to NS4.1 mAb and passed over a 2 ml Sepharose column to which 6F10 mAb was coupled by cyanogen bromide (Pharmacia). The 6F10 specific column was washed with lysis buffer and then eluted with 50 mM diethylamine (pH11) and 0.5 ml fractions were neutralized with 50 μl of 1 M Tris buffer, pH 6.8. The protein containing fractions were pooled, concentrated with Centricon 30 (Amicon), resolved by isoelectric focusing and SDS-PAGE and then electroblotted to a PVDF membrane (BioRad). After the protein was visualized with Ponceau-S, the protein bound membrane was excised and then digested with trypsin. The derived peptides were separated with HPLC and sequenced using an Applied Biosystems model 470 A gas phase sequencer equipped with a model 120A phenylhydantoin amino acid analyzer (Harvard University Micro chemistry Facility, Cambridge, Mass.).

### Example 15

Antibodies

The following mAbs were used as isotype matched negative controls in immunohistochemistry: rat IgG1 (R59-40; Pharmingen, San Diego, Calif.), rat IgG2a (R35-95; Pharmingen), rat IgG2b (SFR3-DR5, anti human HLA-DR5; ATCC, Rockville, Md.), and hamster IgG (UC8-4B3, anti trinitrophenol; Pharmingen). When polyclonal rabbit sera were used, normal rabbit serum served as control. The following mAbs were used to detect murine antigens: anti-CD3e (500A2, hamster IgG, Pharmingen), anti-CD4 (RM4-5, rat IgG2a, Pharmingen), anti-CD8a (53-6.72, rat IgG2a, ATCC), anti-CD45RB (MB23G2, rat IgG2a, ATCC and 16A, FITC-conjugated rat IgG2a, Pharmingen), anti-CD25 (high affinity IL-2 receptor a-chain, 3C7, rat IgG2b, Pharmingen), anti-CD11b (a^{M}-integrin, Mac-1, M1/70, rat IgG2b, ATCC), anti-CD18 (b₂-integrin, 2E6, hamster IgG, ATCC), anti-B220 (RA3-6B2, rat IgG2a, Pharmingen), anti-MHC class II (I-A antigens, M5/114.15.2, rat IgG2b, ATCC), anti-human involucrin (SY5, mouse IgG, Santa Cruz), anti-CD49f (a⁶ integrin, GoH3, rat IgG, Dianova, Hamburg, Germany), anti-MHC class II (N22, hamster IgG, ATCC), anti-CD54 (ICAM-1, YN1/1.7.4, rat IgG2a, ATCC), anti-CD106 (VCAM-1, M/K-2.7, rat IgG1, ATCC), anti-CD31 (PECAM-1, MEC13.3, rat IgG2a, Pharmingen), anti-IFNg (XMG1.2, rat IgG1, Pharmingen), anti-IL-6 (MP5-20F3, rat IgG1, Pharmingen), anti-GM-CSF (MP1-22E9, rat IgG2a, Pharmingen), anti-CD32/CD16 (Fc-gII/III receptor, 2.4G2, rat IgG2b, ATCC), anti-H-2D^{d} (34-2-12, biotinylated C3H IgG2a, Pharmingen), anti H-2K^{b} (AF6-88.5, biotinylated Balb/c IgG2a, Pharmingen). Rabbit sera against murine keratin 6 (Roop, D. R., et al. (1984) J. Biol. Chem., 259:8037-8040; Roop, D. R., et al. (1985) Ann N.Y. Acad. Sci,. 455:426-435), TNFa (#IP-400, Genzyme, Cambridge, Mass.) and IL-1a (#IP-110, Genzyme) also were used. Biotinylated goat-anti-hamster serum and mouse adsorbed rabbit-anti-rat serum were purchased from Vector Laboratories Inc. (Burlingame, Calif.) and goat-anti rat IgG MicroBeads were obtained from Miltenyi Biotec Inc. (Auburn, Calif.).

### Example 16

H-2 Typing

F₂(Balb/c x 129/Sv) donor mice were tail bled and peripheral blood mononuclear cells (PBMC) were isolated by density gradient centrifugation using Histopaque-1083 (Sigma Chemicals, St. Louis, Mo.). The PBMC were incubated with 10 mg/ml anti-FcgRII/III for 10 min. An aliquot of the PBMC from each mouse then was incubated for 30 min with 10 mg/ml of either biotinylated anti-H-2D^{d} (mAb 34-2-12), anti-H-2K^{b} (mAb AF6-88.5), or staining buffer, washed and then incubated with a 1:100 dilution of PE-Streptavidin (Pharmingen), washed and analyzed in a FACSort (Becton Dickinson).

### Example 17

Cell Purification and Reconstitution of SCID-mice

CD4⁺/CD45RB^{hi} and CD4⁺/CD45RB^{1o} T-cells were purified from spleens of Balb/c or F₂(Balb/c x 129/SvJ) mice as described by Powrie et al. (Morrissey, P. J., et al. (1993) J. Exp. Med. 178:237-244; Powrie, F., et al. (1993) Int. Immunol. 5:1461-1471; Powrie, F., et al. (1994) J. Exp. Med. 179:589-600; Morrissey, P. J., et al. (1995) J. Immunol. 154:2678-2686; Powrie, F., et al. (1996) J. Exp. Med. 183:2669-2674) with minor modifications. Spleens from 4-6 donor mice were removed, a single cell suspension was prepared and erythrocytes were lysed by incubation in 0.17 M NH₄Cl for 10 minutes. The cell suspension then was incubated for 15 minutes with 20 mg/10⁷ cells each of azide-free anti B220 (mAb RA3-6B2), anti integrin a^{M} (mAb Mi/70), rat-anti CD8a (mAb 53-6.72) and rat-anti I-A^{b,d,q} (mAb M5/114.15.2), washed twice with 5% FCS in PBS (MACS-buffer), then incubated with 20 ml goat-anti-rat IgG microbeads (Miltenyi Biotec Inc., Auburn, Calif.) per 10⁷ cells for 15 min, and washed again. Cells which did not bind to a MACS separation column (type CS, Miltenyi Biotec Inc.) were collected. The enriched CD4⁺ population (>85% CD4⁺) was incubated with 15 ml PE-conjugated rat-anti CD4 (mAb RM4-5) per 10⁷ cells and 25 ml FITC-conjugated rat-anti CD45RB (mAb 16A) per 10⁷ cells for 30 min, washed and sorted using a FACS Vantage (Becton Dickinson, San Jose, Calif.). From the CD4⁺ population, the 35-40% of cells stained most brightly with anti-CD45RB and the 15-20% of least bright stained cells were selected as CD45RB^{hi} and CD45RB^{1o}, respectively. Each of the collected cell populations was >93% pure. Each recipient scid-mouse was intraveneously injected with either 2.45×10⁵ CD4⁺/CD45RB^{hi} cells, 2.45×10⁵ CD4⁺/CD45RB^{1o} cells, or a mixture of 2.45×10⁵ CD4⁺/CD45RB^{hi} and 0.8×10⁵ CD4⁺/CD45RB^{1o} cells in 300 ml PBS. All purification steps were carried out under sterile conditions at 4 [symbol] C or on ice. In order to remove sodium azide, MicroBeads were pre-run over a separation column and washed twice with MACS buffer.

### Example 18

Clinical Evaluation

Mice were weighed and evaluated clinically at weekly intervals. To more objectively assess the disease development, a clinical score was developed. The ear thickness was determined using a skin thickness gage (“Oditest” from Dyer Inc., Lancaster, Pa. or Fisher Scientific, Pittsburgh, Pa.) at the time of sacrifice.

### Example 19

Histochemistry, Immunohistochemistry and BrdU-labeling

Histological procedures were performed using plastic-embedded tissue. Briefly, tissue samples were fixed in 4% paraformaldehyde at 4° C. overnight and dehydrated 30 min each in 70%, 90%, and 2×30 min in 100% acetone. The samples then were infiltrated and embedded in JB-4 resin according to the manufacturer's instructions (Polysciences Inc., Warrington, Pa.). 1 mm sections were stained with hematoxylin and eosin according to standard protocols. Chloroacetate-esterase staining was performed as described previously (Yam, L. T., et al. (1971) Am. J. Clin. Pathol. 55:283-290. Briefly, prior to each staining new fuchsin solution was prepared by dissolving 1 g new fuchsin (Sigma Inc., St. Louis, Mo.) in 25 ml 2 N HCl and adding an equal volume of freshly prepared 4% NaNO₂. Then, 0.05 ml of the new fuchsin solution and 1 mg naphthol-AS-D-chloroacetate (Sigma) dissolved in 0.5 ml N,N′-dimethyl-formamide (Sigma) were added to 9.5 ml phosphate buffer (0.15 M, pH 7.6). Tissue sections were incubated with the final solution for 10 min at room temperature, rinsed four times with water, counterstained for 2 minutes with 1% methyl green (in 0.1 N sodium acetate, pH 4.2), rinsed with water, and mounted.

For immunohistochemistry, tissue samples were embedded in O.C.T. compound (Miles Inc., Elkhart, Ind.), snap frozen in liquid nitrogen and stored at −20° C. 5 mm cryostat-cut sections were stained by the ABC-immunoperoxidase method (Vector). Briefly, sections were air dried for 30 min, fixed in acetone for 10 min at room temperature, and incubated with buffer containing 30% bovine calf serum, 10% normal goat serum, 5% normal rabbit serum, and 1% normal horse serum for 30 min. Unless otherwise stated, sections then were incubated with 10 mg/ml of the primary antibody for 1 h. After washing with PBS, endogeneous peroxidase was blocked with 0.3% H₂O₂ in PBS for 20 min. Slides were submerged three times for 3 min in PBS and then incubated with biotinylated goat-anti-hamster, mouse adsorbed rabbit-anti-rat, or horse-anti-mouse serum (Vector), according to the primary antibody used. After washing, sections were incubated with the avidin-peroxidase complex according to the manufacturers instructions (Vector) for 45 min, washed with PBS, and submerged in 3-amino-9-ethylcarbazole (red reaction product) or diaminobenzidine (brown reaction product) (both from Sigma) substrate solution in 0.1 M acetate buffer (pH 5.2). Color development was monitored by microscopy, and the reaction stopped by placing the slides in 10% formalin in acetate buffer (pH 5.2) for 10 min. Subsequently, slides were counterstained with hematoxylin, extensively washed with water, incubated 3 min in a saturated solution of LiCO3, washed, and mounted with Gel/Mount (Biomeda Corp., Foster City, Calif.). All steps were carried out at room temperature.

In order to detect proliferating cells, 3 uninjected mice and 3 mice injected with CD4⁺/CD45RB^{hi} T-cells were injected intraperitoneally with 5 mg BrdU in 500 ml PBS at both 9 and 6 h prior to sacrifice. 4 mm paraffin-sections were immersed in 0.03% H₂O₂ in methanol for 30 min and washed with TBS. Sections were denatured by incubation with 0.4% pepsin (Sigma) in 0.1 N HCl for 20 min at 37° C. and then 0.8 N HCl for 20 min at room temperature. Sections then were stained by the ABC-immunoperoxidase method (Vector) as described above using an anti-BrdU mAb (Becton Dickinson).

### Example 20

Immunohistochemistry and Flow Cytometry (FACS)

Immunohistochemistry was performed on acetone-fixed 5 μm cryostat-cut sections using 10 μg/ml of primary antibody. Antibody reactivity was visualized by the ABC immunoperoxidase method (Vector Laboratories, Burlingame, Calif.) according to the manufacturer's instructions using 3-amino-9-ethylcarbazole as chromogen. Stained slides were fixed in 4% formalin, and counterstained with hematoxylin and LiCO₃.

For double-labeling, sections were incubated with 10 μg/ml of 6F10 mAb (the first primary antibody) followed by 1:50 diluted FITC-conjugated anti-mouse-antibody. Sections then were incubated with 10 μg/ml of biotinylated second antibody (specific for CD1 a, or MHC class II) followed by the ABC immunoperoxidase method as described above. 6F10 reactivity then was assessed in the fluorescent mode, and reactivity for the other antigens was assessed in the regular light mode using a Nikon fluorescence microscope. An exception was made when anti-CD 14 mAbs were used, as these reagents did not work in immunohistochemistry in a biotinylated form. In this case, cryostat-cut sections were incubated with purified mAb 6F10 followed by anti-CD 14 mAbs. Antibody binding then was detected using FITC-conjugated anti-mouse-IgG (for antiCD14 staining) followed by phycoerythrin-conjugated anti-mouse-IgM (to detect mAb 6F10 staining).

For FACS-analysis, 105 cells were incubated in staining buffer (2% bovine serum albumin and 5% goat serum in PBS). Thereafter, cells were incubated with saturating amounts of primary antibody in staining buffer followed by 1:50 diluted FITC-conjugated secondary antibody. Cells were analyzed using a FACSort (Becton Dickinson) and the Cell Quest software.

### Example 21

Modified Stamper-Woodruff Assays

Five μm cryostat-cut sections of normal or psoriatic human skin were mounted on pre-cleaned slides, air dried, and surrounded by a hydrophobic barrier (Pap-Pen, Immunotech). Sections then were overlayered with 20% FCS in PBS and incubated twice for 15 minutes at 37° C. For antibody blocking, sections then were incubated with 1:2 0 diluted ascites or 20 μg/ml of purified mAb for 30 minutes at 37° C. While the sections were blocking, PHA-blasts were washed twice in RPMI1640 supplemented with 10% FCS and 15 mM HEPES, and resuspended at 106 cells/ml. The medium was pre-incubated for at least 1 hour at 37° C. and 5% CO₂. Sections then were overlayered with equal volumes of cell suspension (10⁶ cells/ml) and incubated for 35 minutes at 37° C. and 5% CO₂. Thereafter, slides were washed 5× in PBS, fixed in 8% formalin for 10 minutes, washed twice in deionized water, and counterstained with hematoxylin and LiCO₃. Cells bound to the skin sections were quantitated per mm epidermis using a 20× lens.

### Example 22

T cell Migration Assays into Keratinocyte Monolayers

Directed haptotactic T cell migration was studied using modified Boyden chambers as described (Schön, M., et al. (1996) J. Invest. Dermatol. 106:1175-1181) with the following modifications: Polycarbonate filters with 8 μm pore size (Costar) were over layered with a single cell suspension of the spontaneously immortalized human keratinocyte line HaCaT (Boukamp, P., et al. (1988) J. Cell Biol. 106:761-771) and incubated for 3.5 hours at 37° C. and 5% CO₂. Confluency of the HaCaT monolayer was confirmed on representative filters by hematoxylin staining and subsequent microscopic examination. Filters then were placed upside-down into the chambers (Costar) and equilibrated in lymphocyte culture medium at 37° C. and 5% C02 for 1 hour. For antibody blocking studies, 20 μg/ml of mAb was added to both compartments for at least 30 minutes. While the Boyden chambers were equilibrating, PHA-blasts were washed twice in serum-free medium and stained with the red-fluorescent intravital dye PKH26-GL (Sigma, St. Louis, Mo.) according to the manufacturer's instructions. Briefly, cells were resuspended in diluent (0.5 ml/10⁷ cells) and an equal volume of 1:250 diluted PKH26-GL (working concentration 2×10⁻⁶ M) was added for 5 minutes at room temperature. The reaction was stopped by adding FCS (1 ml/10⁷ cells) for 1 minute. The cells then were washed twice in culture medium preincubated at 37° C. and 5% CO₂, and resuspended at 5×10⁵ cells/150 μl. Viability of stained cells was confirmed by trypan blue exclusion and was generally greater than 95%, and effective labeling was confirmed by fluorescence microscopy. Labeled PHA-blasts (5×10⁵ cells/150 μl) then were added to the upper compartment of the Boyden-chamber and allowed to migrate for 3.5 hours. Uncoated filters were used to assess unspecific binding. Filters then were removed from the chambers, washed 5× in PBS in a standardized fashion, fixed in 8% formalin, and mounted onto slides. Three representative filters were embedded in O.C.T., snap-frozen in liquid nitrogen, and 5 μm cryostat-cut cross-sections were analyzed in a fluorescent microscope to confirm migration of PHA-blasts into the HaCaT monolayer. For each filter, the number of migrated PHA-blasts in at least 12 microscopic fields was determined by a blinded observer under a fluorescent microscope using a 40× lens and the counts were averaged. The experiments were performed in triplicates and the data were expressed as the mean of migrated cells/mm² (+SD).

### Example 23

T Cell Migration into Multilayered Organotypic Keratinocyte Cultures

The organotypic cultures were placed upside-down on a sterile Petri dish, and collagen/fibroblast-matrix was easily peeled off the organotypic cultures of human keratinocytes (strain N). The integrity of the remaining stratified epithelium was confirmed by hematoxylin-stained cryostat-cut sections of representative cultures.

The epidermis equivalents then were soaked in lymphocyte culture medium containing 20 μg/ml of mAb. Surface binding of mAb was confirmed by direct immunofluorescence using both cryostat-cut cross-sections and whole-mount cultures. While the epidermal sheets were incubating, PHA-blasts or the skin-derived T cell line TSBR-1 were intravitally labeled with PKH26-GL as outlined above. Organotypic cultures then were placed upside-down into 24-well tissue culture plates and equal volumes of labeled T cells were added to the surface. After a sedimentation period of 10 minutes, cultures were overlayered with 500 μl of lymphocyte culture medium containing blocking or control mAb and incubated for 3.5 hours at 37° C. and 50% CO₂. Cultures then were washed 5 times in PBS in a standardized fashion and mounted onto microscope slides. Representative cultures from all experiments were snap-frozen in liquid nitrogen and cryostat-cut sections were used to confirm T cell migration into suprabasal epidermal layers. Whole-mounts were used to quantitate migrated T cells in a blinded fashion as outlined above. The experiments were performed in triplicates and the data were expressed as the mean of migrated cells/mm² (±SD).

### Example 24

Statistical Analysis

Statistical significance was assessed by paired two-tailed Student'sT-test.

### Example 25

Generation of LEEP-CAM Specific Monoclonal Antibodies

LEEP-CAM specific monoclonal antibodies were generated by immunizing Balb/c mice with purified LEEP-CAM. LEEP-CAM was immunoisolated as follows: 2×10⁹ 16E6.a5 epithelial cells were solubilized for 1 hour on ice in 1% Triton X-100 in Tris buffered saline (TBS, 10 mM Tris, 150mM NaCl, pH 8.0) containing the protease inhibitors iodoacetamide and phenylmethylsulfonyl fluoride and their nuclei pelleted. The lysates were clarified by centrifugation at 100,000×g for one hour and applied successively to a mouse IgM column and LEEP-CAM specific 6F10 mAb column. After extensive wash with a buffer containing 0.5% Sodium Deoxycholate, 0.05% SDS and 0.5% Triton X-100 in TBS, LEEP-CAM was eluted by 50 mM diethylamine (pH 11) and the fractions neutralized with 1M Tris, pH 6.8. The fractions were assayed for the presence of LEEP-CAM by SDS-PAGE and silver staining. Positive fractions were pooled, concentrated by ethanol precipitation followed by lyophilization and resuspended in water. Three subcutaneous injections of LEEP-CAM emulsified in Freund's adjuvant was give at 3-4 week intervals. Four days prior to fusion, the last injection of LEEP-CAM was given intraperitoneally. On the day of fusion, splenocytes were isolated, fused with P3X63Ag8.653 myeloma cells in the presence of 50% PEG and the hybridomas selected as per standard protocol. Hybridoma supernatants were screened by western blotting for their ability to detect LEEP-CAM in the membranes of 16E6.A5 cells. The selected hybridomas were subcloned two times by limiting dilution and characterized further.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a LEEP-CAM mediated disorder in a mammal without depleting lymphocytes in the mammal comprising administering to the mammal a therapeutically effective amount of an anti-LEEP-CAM compound.

2. The method of claim 1 wherein the anti-LEEP-CAM compound is a small molecule.

3. The method of claim 1 wherein the anti-LEEP-CAM compound is an antibody.

4. The method of claim 1 wherein the disorder is selected from the group consisting of psoriasis, asthma, eczema, T cell tumors which infiltrate skin, arthritis, Rheumatoid arthritis, Graft vs. Host disease, local infections, dermatoses, inflammatory bowel diseases, autoimmune diseases, lichen ruber planus, Crohn's disease, and ulcerative colitis.

5. The method of claim 1 wherein the mammal is a human.

6. The method of claim 1 wherein the administration is cutaneous, mucosal or parenteral.

7. An antibody which is an anti-LEEP-CAM antibody.

8. The antibody of claim 7 which is a polyclonal antibody, monoclonal antibody, an antibody fragment, or a mimitope.

9. A monoclonal antibody according to claim 8 which is a 6F10 monoclonal antibody.

10. A monoclonal antibody according to claim 8 which binds to a 90-115 kDa or a 145 kDa molecule which is expressed constitutively in the suprabasal epidermal layers of a mammal and which modulates migration of T lymphocytes into an epithelial layer of the mammal.

11. A method for preventing or modulating skin inflammatory disorders in a mammal by administering to the mammal a therapeutically-effective amount of a substance which prevents LEEP-CAM-mediated migration of T lymphocytes into an epithelial cell layer.

12. A method of preventing or treating a LEEP-CAM mediated disorder in a mammal without depleting T lymphocytes in the mammal comprising administering to the mammal a therapeutically effective amount of a compound which binds to a LEEP-CAM ligand on a T cell.

13. A method of diagnosing a disorder or disease mediated by LEEP-CAM comprising:
a) detecting anti-LEEP-CAM antibody binding to LEEP-CAM positive cells taken in a sample from a subject; and
b) diagnosing the medical condition on the basis of such binding.

14. A diagnostic kit according to claim 13 which contains:
a) an antibody with specificity for LEEP-CAM, or a biologically active derivative of the antibody, preferably labeled with a substance which permits detection of binding of the antibody to LEEP-CAM; and
b) purified LEEP-CAM, to provide a standard for evaluation of the assay results.

15. The kit of claim 14, wherein the antibody is a monoclonal antibody.

16. A therapeutic composition comprising a therapeutically effective amount of a modulator of LEEP-CAM function in a pharmaceutically acceptable carrier.

17. The therapeutic composition of claim 16 wherein said modulator is an inhibitor of LEEP-CAM function.

18. The therapeutic composition of claim 16 wherein said modulator upregulates LEEP-CAM function.

19. The therapeutic composition of claim 16 wherein said modulator interferes with the interaction of T lymphocytes and LEEP-CAM.

20. The therapeutic composition of claim 19 wherein said modulator is a small molecule, an antibody, or a monoclonal antibody.

21. A method of treating a mammal to decrease or prevent an inflammatory response, the method comprising:
a) identifying an area of the mammal having a local inflammatory response; and
b) administering a therapeutic composition comprising a LEEP-CAM inhibitor in a therapeutically effective amount to the area of local inflammatory response, whereby LEEP-CAM molecules are unable to interact with lymphocytes in the area of local inflammatory response, whereby the inflammatory response is decreased.

22. The method of claim 21 wherein the area of local inflammatory response is selected from the group consisting of suprabasal region of the epidermis, the basal layer of bronchial epithelia, the basal layer of breast epithelia, the tonsillar epithelia, the vaginal epithelia, the vascular epithelium, and the high endothelial venule endothelia.

23. The method of claim 21 wherein the lymphocyte is a T lymphocyte.

24. The method of claim 21 wherein the lymphocyte is a B lymphocyte.

25. A 90-115 kDa cell surface glycoprotein which binds to a 6F10 monoclonal antibody and which is expressed constitutively in the suprabasal epidermal layers of a mammal.

26. Use of an anti-LEEP-CAM compound for the manufacture of a medicament for treating (for example by cutaneous, mucosal or parenteral administration) a LEEP-CAM mediated disorder in a mammal, e.g. a human, without depleting lympocytes in the mammal.

27. The use of claim 26 wherein the anti-LEEP-CAM compound is a small molecule.

28. The use of claim 27 wherein the anti-LEEP-CAM compound is an antibody.

29. The use of claim 26 wherein the disorder is selected from the group consisting of psoriasis, asthma, eczema, T cell tumors which infiltrate skin, arthritis, Rheumatoid arthritis, Graft vs. Host disease, local infections, dermatoses, inflammatory bowel diseases, autoimmune diseases, lichen ruber planus, Crohn's disease, and ulcerative colitis.

30. An antibody for use in therapy or in vivo diagnosis which is an anti-LEEP-CAM antibody.

31. The antibody of claim 30 which is a polyclonal antibody, monoclonal antibody, an antibody fragment, or a mimitope.

32. A monoclonal antibody according to claim 31 which is a 6F10 monoclonal antibody.

33. A monoclonal antibody according to claim 31 which binds to a 90-115 kDa or a 145 kDa molecule which is expressed constitutively in the suprabasal epidermal layers of a mammal and which modulates migration of T lymphocytes into an epithelial layer of the mammal.

34. Use of a substance which prevents LEEP-CAM-mediated migration of lymphocytes into an epithelial cell layer, for the manufacture of a medicament for preventing or modulating skin inflammatory diseases in a mammal.

35. Use of a compound which binds to a LEEP-CAM ligand on a T cell for the manufacture of a medicament for preventing or treating a LEEP-CAM mediated disorder in a mammal without depleting T lymphocytes in the mammal.

36. Use of a LEEP-CAM inhibitor for the manufacture of a medicament for treating or preventing disease in a mammal by decreasing an inflammatory response, by
a) identifying an area of the mammal having a local inflammatory response; and
b) administering the medicament comprising the LEEP-CAM inhibitor in a therapeutically effective amount to the area of local inflammatory response, whereby LEEP-CAM molecules are unable to interact with lymphocytes in the area of local inflammatory response, whereby the inflammatory response is decreased.

37. The use of claim 36 wherein the area of local inflammatory response is selected from the suprabasal region of the epidermis, the basal layer of bronchial epithelia, the basal layer of breast epithelia, the tonsillar epithelia, the vaginal epithelia, the vascular epithelium, or the high endothelial venule endothelia.

38. The use of claim 36 wherein the lymphocyte is a T lymphocyte or a B lymphocyte.

39. Use of a 90-115 kDa cell surface glycoprotein which binds to a 6F10 monoclonal antibody and which is expressed constitutively in the suprabasal epidermal layers of a mammal, for the manufacture of a medicament for treating a LEEP-CAM mediated disorder in a mammal.

40. Use according to claim 39 wherein the disorder is selected from the group consisting of psoriasis, asthma, eczema, T cell tumors which infiltrate skin, arthritis, Rheumatoid arthritis, Graft vs. Host disease, local infections, dermatoses, inflammatory bowel diseases, autoimmune diseases, lichen ruber planus, Crohn's disease, and ulcerative colitis.

41. Use of an antibody as defined in any one of claims 30-33 in in vitro diagnosis.

42. Use of a compound for the manufacture of a medicament for upregulating (for example by cutaneous, mucosal or parenteral administration) a disorder in a mammal, e.g. a human, which disorder results from lack of LEEP-CAM presence or expression.
